(19) United States
(12) Patent Application Publication
(10) Pub. No.: US 2020/0316072 A1
(43) Pub. Date: Oct. 8, 2020

(54) COMPOUNDS FOR USE IN REGULATING FOLLICLE MATURATION

(71) Applicant: AARHUS UNIVERSITET, Aarhus C (DK)

(72) Inventors: Karin Lykke-Hartmann, Skødstrup (DK); Emil Hagen Ernst, Silkeborg (DK); Anders Heuck, Skanderborg (DK)

(73) Assignee: AARHUS UNIVERSITET, Aarhus C (DK)

(21) Appl. No.: 16/955,136

(22) PCT Filed: Dec. 20, 2018
(86) PCT No.: PCT/EP2018/086239

§ 371 Date: Jun. 18, 2020

(30) Foreign Application Priority Data
Dec. 21, 2017 (EP) 17209250.4

(51) Int. Cl. A61K 31/517; A61K 31/407; A61K 31/575; A61K 31/18; A61K 31/454; A61K 31/522; A61K 31/4985; A61K 31/69; A61K 31/573; A61K 31/4402; A61K 31/5377; A61K 31/502; A61K 31/4439; A61K 31/501

(52) U.S. Cl. CPC A61K 31/517; A61K 45/06; A61K 31/575; A61K 31/18; A61K 31/454; A61K 31/522; A61K 31/4985; A61K 31/69; A61K 31/573; A61K 31/4402; A61K 31/5377; A61K 31/502; A61K 31/4439; A61K 31/501; A61K 31/407

(57) ABSTRACT

The present invention relates to a compounds and methods for regulating follicle maturation, and the use thereof in treating, preventing or ameliorating an ovulation disorder and methods for treating females having an ovulation disorder.

### TECHNICAL FIELD

The present invention relates to a method for screening for a compound that regulates follicle maturation, compounds that regulates follicle maturation, for use in treating, preventing or ameliorating an ovulation disorder and methods for treating females having an ovulation disorder.

### BACKGROUND

The ability to conceive a child often seems like the most natural and frequent occurrence, representing an event that is required to main the continuous development of our society. However, there is a large and increasing group of infertile women, who cannot be helped with the current treatment. These women represent a major unmet medical need where novel treatment strategies are urgently required.

The reproductive capacity of women depends on several factors, e.g. genetics, environment and age. The pool of the earliest primordial (resting) follicles is almost completely laid down in ovaries during fetal life and constitutes at any moment in time the reproductive potential of a female. The fertility treatment today is directed towards the later stages of follicle development, and if women can produce secondary follicle, this treatment usually is very effective. However, a large fraction of women remains infertile, due to the fact that primordial (resting) follicles are not activated.

Activation of primordial follicles is marked morphologically by the primordial-to-primary transition when the flattened granulosa cells start proliferation and become cuboidal and the oocyte grows in size. The primordial-to-primary follicle transition is a gradual process in which intermediate follicles with both flattened and cuboidal granulosa cells can be noted. Maintaining dormancy of primordial follicles on one hand, and gradual activation towards ovulation on the other, is a tightly regulated process involving several pathways. Activators and inhibitors of the primordial-to-primary follicle transition are known to be important for its regulation.

Currently, our knowledge regarding the molecular pathways that drives this follicle transition is sparse. Cellular signalling pathways including PI3K/AKT, AKT/mTOR and HIPPO, as well as TGF-beta and IGF signalling are known to regulate the primordial-to-primary transition in mammalian follicle development. Pathway activators (Kit ligand, FGF-2, KGF, LIF, BMP-417, 15 and GDF-9) and suppressors (PTEN, Tsc1m/TORC1, FOXO3a, Fox12, p27, LHX8, FOXL2 and AMH) of the primordial-to-primary follicle transition are known to be important for its regulation.

For example, conditional ablation of FOXO3a, PTEN, and Tsc1/2 in oocytes triggers increased oocyte activation (Also, in vitro culture of human ovarian tissue with PTEN inhibition, and AKT stimulation has been shown to increase primordial follicle activation. Studies on human oocytes have been performed on a pool of isolated oocytes from primordial, intermediate and primary follicles or in combination with the surrounding granulosa cells.

Ovarian deficits and consequently infertility are a growing problem worldwide and is often attributed to compromised or poor egg production in females. In one instance, the women remain infertile, due to the fact that primordial (resting) follicles are not activated. Pushing forward egg maturation may help women with for example age related decline in eggs and/or women with pathologies such as for example Polycystic Ovary Syndrome. In another instance, the major challenge is to protect the pool of resting eggs, i.e. the reproductive potential, from premature expiration. Thus, holding back egg maturation may help women who need to maintain an egg pool, for example during disease treatment, such as cancer treatment, or due to genetic diseases or disorders causing premature depletion of eggs. Therefore, there is a great need to control the most important early steps of egg development.

### SUMMARY

The inventors of the present invention have identified compounds for use in regulating follicle maturation, in particular for use in regulating the primordial to primary transition of follicles. Transcriptome dynamics specifically associated with human granulosa cells from primordial and primary follicles, respectively, have been characterized and used to identify targets and screen for potential compounds that can regulate follicle maturation, in particular early stages of follicle maturation such as the primordial to primary transition.

Accordingly, aspect of the present invention relates to a method for screening for a compound that regulates follicle maturation, wherein said method comprises
a. selecting a compound known to regulate the activity of at least one of the candidates identified in table 3.
b. contacting a primordial and/or a primary ovary with said compound
c. determining whether said compound is capable of regulating follicle maturation by determining the amount of primordial follicles and/or primary follicles and compare it with a control.

The method has been used to identify compounds described below.

Thus, one aspect of the present invention relates to a compound for use in regulating follicle maturation, wherein said compound can regulate the activity of Dipeptidyl Peptidase 4. In one embodiment said compound is an inhibitor of Dipeptidyl Peptidase 4. Preferably, said inhibitor inhibits follicle maturation. In a preferred embodiment, said inhibitor is selected from the group consisting of Saxagliptin, Alogliptin, Sitagliptin, Vildagliptin, Linagliptin and Talabostat.

Another aspect of the present invention relates to a compound for use in regulating follicle maturation, wherein said compound can regulate the activity of toll like receptor 3. In one embodiment said compound is an inhibitor of toll like receptor 3. Preferably, said inhibitor inhibits follicle maturation. In a preferred embodiment, said inhibitor inhibitor is Rintatolimod.

A further aspect of the present invention relates to a compound for use in regulating follicle maturation, wherein said compound can regulate the activity of gamma-glutamyl carboxylase. In one embodiment said compound is an inhibitor of gamma-glutamyl carboxylase. Preferably, said inhibitor inhibits follicle maturation. In a preferred embodiment, said inhibitor is selected from the group consisting of Anisindione and Menadione.

Another aspect of the present invention relates to a compound for use in regulating follicle maturation, wherein said compound can regulate the activity of glutamate metabotropic receptor 8. In one embodiment said compound is an activator of glutamate metabotropic receptor 8. Preferably, said activator stimulates follicle maturation. In a preferred embodiment, said activator is Fasoracetam.

Yet another aspect of the present invention relates to a compound for use in regulating follicle maturation, wherein said compound can regulate the activity of smoothened, frizzled class receptor (SMO). In one embodiment said compound is an inhibitor of smoothened, frizzled class receptor. Preferably, said inhibitor inhibits follicle maturation. In a preferred embodiment, said inhibitor is selected from the group consisting of NVP-LEQ-506, TAK-441, PF-04449913, Taladegib, Sonidegib, Saridegib and Vismodegib.

A further aspect of the present invention relates to a compound for use in regulating follicle maturation, wherein said compound can regulate the activity of potassium voltage-gated channel subfamily B member 1 (KCNB1). In one embodiment said compound is an inhibitor of KCNB1. Preferably, said inhibitor stimulates follicle maturation. In a preferred embodiment, said inhibitor is Dalfampridine.

A further aspect of the present invention relates to a compound for use in regulating follicle maturation, wherein said compound can regulate the activity of Prolactin receptor. In one embodiment said compound is an inhibitor of Prolactin receptor. Preferably, said inhibitor inhibits follicle maturation. In a preferred embodiment, said inhibitor is Fluoxymesterone.

Another aspect of the present invention relates to a compound for use in regulating follicle maturation, wherein said compound can regulate the activity of cyclin dependent kinase 1. In one embodiment said compound is an inhibitor of cyclin dependent kinase 1. Preferably, said inhibitor stimulates follicle maturation. In a preferred embodiment, said inhibitor is selected from the group consisting of Dinaciclib, Milciclib, Roniciclib and Alvocidib.

Yet another aspect of the present invention relates to a compound for use in regulating follicle maturation, wherein said compound can regulate the activity of neurotrophic receptor tyrosine kinase 2. In one embodiment said compound is an inhibitor of neurotrophic receptor tyrosine kinase 2. Preferably, said inhibitor inhibits follicle maturation. In a preferred embodiment, said inhibitor is selected from the group consisting of Cabozantinib, Theophylline and Entrectinib.

A further aspect of the present invention relates to a compound for use in regulating follicle maturation, wherein said compound can regulate the activity of phospholipase C like 1. In one embodiment said compound is an inhibitor of phospholipase C like 1. Preferably, said inhibitor inhibits follicle maturation. In a preferred embodiment, said inhibitor is Quinacrine.

Another aspect of the present invention relates to a compound for use in regulating follicle maturation, wherein said compound can regulate the activity of gamma-aminobutyric acid type A receptor epsilon subunit. In one embodiment said compound is an activator of gamma-aminobutyric acid type A receptor epsilon subunit. Preferably, said activator stimulates follicle maturation. In a preferred embodiment, said activator is selected from the group consisting of Eszopiclone and Pregnenolone.

A further aspect of the present invention relates to a compound for use in regulating follicle maturation, wherein said compound can regulate the activity of protein tyrosine phosphatase, non-receptor type 1. In one embodiment said compound is an inhibitor of protein tyrosine phosphatase, non-receptor type 1. Preferably, said inhibitor inhibits follicle maturation. In a preferred embodiment, said compound according to any of claims 46-47, wherein said inhibitor is Trodusquemine.

A further aspect of the present invention relates to a compound for use in regulating follicle maturation, wherein said compound can regulate the activity of xanthine dehydrogenase. In one embodiment said compound is an inhibitor of xanthine dehydrogenase. Preferably, said inhibitor inhibits follicle maturation. In a preferred embodiment, said inhibitor is selected from the group consisting of Allopurinol, Oxypurinol and Febuxostat.

Another aspect of the present invention relates to a compound for use in regulating follicle maturation, wherein said compound can regulate the activity of WEE1 G2 checkpoint kinase. In one embodiment said compound is an inhibitor of WEE1 G2 checkpoint kinase. Preferably, said inhibitor inhibits follicle maturation. In a preferred embodiment, said inhibitor is MK 1775.

Another aspect of the present invention relates to a compound for use in regulating follicle maturation, wherein said compound can regulate the activity of protein kinase C beta. In one embodiment said compound is an inhibitor of protein kinase C beta. Preferably, said inhibitor inhibits follicle maturation. In a preferred embodiment, said inhibitor is selected from the group consisting of Sotrastaurin, Enzastaurin and Ruboxistaurin.

Yet another aspect of the present invention relates to a compound for use in regulating follicle maturation, wherein said compound can regulate the activity of arachidonate 12-lipoxygenase, 12S type. In one embodiment said compound is an inhibitor of arachidonate 12-lipoxygenase, 12S type. Preferably, said inhibitor inhibits follicle maturation. In a preferred embodiment, said inhibitor is selected from the group consisting of Sulfasalazine, Balsalazide, Mesalamine and Masoprocol.

A further another aspect of the present invention relates to a compound for use in regulating follicle maturation, wherein said compound can regulate the activity of amine oxidase copper containing 3. In one embodiment said compound is an inhibitor of amine oxidase copper containing 3. Preferably, said inhibitor inhibits follicle maturation. In a preferred embodiment, said inhibitor is Hydralazine.

Another aspect of the present invention relates to a compound for use in regulating follicle maturation, wherein said compound can regulate the activity of solute carrier family 52 member 2. In one embodiment said compound is an inhibitor of solute carrier family 52 member 2. Preferably, said inhibitor inhibits follicle maturation. In a preferred embodiment, said inhibitor is 4-hydroxybutanoic acid.

Another aspect relates to a compound as described herein and above are for use in regulating follicle maturation in vitro or in vivo.

In another embodiment, the compounds as described herein for use in regulating follicle maturation in vivo.

In a preferred embodiment of the present invention, the compounds are for use in regulating primordial to primary transition of follicles. Preferably, said follicles are mammalian follicles. It is preferred that said mammalian is a human.

A further aspect of the present invention relates to a compound as defined herein for use in treating, preventing or ameliorating an ovulation disorder.

In one embodiment said ovulation disorder is selected from the group consisting of Polycystic ovary syndrome (PCOS), Premature ovarian failure (P01), Hypothalamic dysfunction and Menopause.

In another embodiment said ovulation disorder is caused by hyperprolactinemia.

In another aspect, the present invention relates to a pharmaceutical composition comprising at least one compound as defined herein for use in treating, preventing or ameliorating an ovulation disorder. The ovulation disorder is as defined herein.

Yet another aspect of the present invention relates to a pharmaceutical composition comprising at least one compound as defined herein for use in treating, preventing or ameliorating infertility or reduced fertility in a female individual.

Preferably, said pharmaceutical composition further comprises at least one pharmaceutically acceptable carrier. In another embodiment said pharmaceutical composition further comprises an additional active agent.

Said pharmaceutical composition may for example be administered in vitro to primordial follicles from said individual.

In one preferred embodiment said female individual is a female mammal, preferably a female human.

Another aspect of the present invention relates to a method for treating, preventing or ameliorating infertility of a female individual having an ovulation disorder, comprising administering to said individual a therapeutically effective amount of a compound as defined herein.

### DESCRIPTION OF DRAWINGS

FIG. 1. Experimental design for Laser Capture Microdissection and sequencing. A. Process of Laser Capture Microdissection. Primordial and primary follicles were identified based on morphology, cut, and isolated using Laser Capture Microdissection. Scale bars: 30 μm. For isolation of oocytes from same follicular stages, please refer to (Ernst et al., Hum Reprod. 2017 Aug. 1; 32(8):1684-1700). B. Isolates made sequencing, mapping, annotation and filtration. Pure pools of oocytes from primordial and primary follicles as well as whole follicles from three patients were isolated via Laser Capture Microdissection based on morphological appearance. A total of 12 samples were included for library preparation and sequencing on the Illumina platform. Following alignment to human reference genome (hg19), gene expressions were normalized, transformed to log 2 (CPM) and fragments per kilobase of exon per million fragments mapped (FPKM) was calculated.

FIG. 2. In silico extraction of granulosa cell transcriptome SSCEGs and DEGs lists. In silico extraction of the transcripts originating from the granulosa cells of the follicular compartment were performed by applying a number of filters on NGS data from oocytes of primordial and primary follicles, and whole primordial and primary follicles. *All transcripts unique to whole follicle as compared to oocyte+all transcripts with FPKM value>1.5 unique to whole follicle as compared to oocyte. T-test across triplicates p<0.2 for consistency in gene expression. ** p<0.05 and/or fold-change>2 between primordial follicle granulosa cell transcript and primary follicle granulosa cell transcript. Submitted for enrichment analysis: * and **.

Total transcripts in granulosa cells from primordial follicles included a number of known granulosa cell markers: AR, YY1, ZFX, CLGN, FOXL2, SLC35G1, and FOG2. Total transcripts in granulosa cells from primary follicle included a number of known granulosa cell markers: KITL, FOG2, and ZFX. In both the granulosa cell contribution from primordial and primary follicles we noted absence of oocyte markers such as: FIGLA, NOBOX, ZAR1, MATER (NLRP5), KIT, VASA (DDX4), and GDF-9 which were all present in transcripts identified in both pure isolates of oocytes and oocyte with surrounding granulosa cells (FPKM>1.5) or in oocyte isolates only.

### DETAILED DESCRIPTION

### Definitions

The term “ovary” as used herein refers to the ovum-producing reproductive organ, which is the site of production and periodical release of egg cells, the female gametes.

In the ovaries, the developing egg cell (or oocyte) grows within the environment provided by follicles.

The term “follicle” as used herein refers to a roughly spheroid cellular aggregation set found in the ovaries. The follicle is an anatomical structure in which the oocyte develops surrounded by granulosa cells, which shape and layers depends on the follicle stage. Follicles are composed of different types and number of cells according to the stage of their maturation, and their size is indicative of the stage of oocyte development.

The term “primordial follicle” as used herein refers to the follicles that are formed in the female ovary at 18-22 weeks post-conception. The primordial follicles contain immature oocytes surrounded by flat, squamous granulosa cells (the support cells) that are segregated from the oocyte's environment by the basal lamina. They are quiescent, showing little to no biological activity.

The term “primary follicle” as used herein refers to the follicle that are formed when the granulosa cells of these primordial follicles change from a flat to a cuboidal structure, marking the beginning of the primary follicle. Both the oocyte and the follicle grow dramatically, increasing to almost 0.1 mm in diameter. A glycoprotein polymer capsule called the zona pellucida forms around the oocyte, separating it from the surrounding granulosa cells.

The term “primordial oocyte” as used herein refers to an oocyte from a primordial follicle.

The term “primary oocyte” as used herein refers to an oocyte from a primary follicle.

The term “primordial to primary transition of follicles” as used herein refers to the stage at which the follicle develops or matures from a primordial follicle into a primary follicle.

Thus, the primordial to primary transition is the process by which primordial follicle are activated and matures or develops into a primary follicle. For example, when a compound promotes the primordial to primary transition of follicles, it is meant that the compound promotes or activates the transition of primordial follicles into primary follicles. When a compound inhibits the primordial to primary transition of follicles, it is meant that the compound inhibits the transition of primordial follicles into primary follicles or simply keeps the primordial follicles in the dormant stage.

The terms “regulating follicle maturation” or “controlling follicle maturation” can be used interchangeably and includes preferably activation, stimulation and inhibition of follicle maturation.

Granulosa cells are cells surrounding the oocyte as it develops within the ovary follicle. Layers of granulosa cells are bound to a thick specialised extracellular matrix called the zona pellucida. In the ovarian primary follicle, and later in follicle development, granulosa cells advance to form a multilayered cumulus oophorus surrounding the oocyte in the preovulatory or antral follicle.

### Method for Screening for Compounds

One aspect of the present invention relates to a method for screening for a compound that regulates follicle maturation, wherein said method comprises
d. selecting a compound known to regulate the activity of at least one of the candidates identified in table 3.
e. contacting a primordial and/or a primary ovary with said compound
f. determining whether said compound is capable of regulating follicle maturation by determining the amount of primordial follicles and/or primary follicles and compare it with a control.

Methods for determining whether said compound is capable of regulating follicle development are described in the example section.

In one embodiment the compound to be tested is dissolved in suitable solvent and added to a culture medium comprising primordial and/or primary ovaries. As a control, solvent not comprising the compound can be added to the culture medium.

The ovary can be a mammalian ovary such as for example a mouse ovary or a human ovary.

The compound may also be contacted with a biopsy from an ovary, in particular a biopsy from a human ovary.

The concentration of the compound in the culture medium depends on the compound to be tested. In one embodiment, the concentration of compound is at least 0.01 micromolar (μM), such as at least 0.1 μM, at least 0.5 μM, at least 1 μM or at least 5 μM.

In one embodiment the concentration of compound is between 0.01 μM and 100 millimolar (mM), such as between 0.01 μM and 50 mM or such as between 0.1 μM and 50 mM.

The incubation time may for example be at least 10 hours, at least 24 hours, at least 48 hours, at least 72 hours or at least 96 hours. In one embodiment the incubation time is 1 day, 2 days, 3 days, 4 days or 5 days. In one embodiment the incubation time is in the interval from 1 to 10 days, such as from 1 to 9 days, from 1 to 8 days or preferably from 1 to 7 days.

The primordial and/or primary ovaries are preferably incubated with the compound at 37° C. In particular, the primordial and/or primary ovaries are preferably incubated with the compound at 37° C. and 5% CO₂.

The number and distribution of primordial and primary follicles can be determined using Hematoxylin and eosin (HE) staining and sectioning followed by microscopy to stereologically count the distribution of primordial primary and secondary follicles. The secondary follicels are also included in order to statistically account for the follicles stages. Please see experimental section for further details.

### Compounds for Use in Regulating Follicle Maturation

Compounds identified by the method described above are discussed below.

### Dipeptidyl Peptidase 4 as a Target

One aspect of the present invention relates to a compound for use in regulating follicle maturation, wherein said compound can regulate the activity of Dipeptidyl Peptidase 4 (DPP4).

DPP4 was found to be significantly up-regulated in granulosa cells from human primordial and primary follicles (see Table 3)

When inhibition of follicle maturation is preferred, said compound is preferably an inhibitor of DPP4. Thus, in one embodiment said compound is an inhibitor of DPP4. In a preferred embodiment said compound or inhibitor of DPP4 regulates the primordial to primary transition of follicles. In a particular preferred embodiment said compound or said inhibitor of DPP4 inhibits the primordial to primary transition of follicles.

In one particular preferred embodiment, said compound is an inhibitor selected from the group consisting of Saxagliptin, Alogliptin, Sitagliptin, Vildagliptin, Linagliptin and Talabostat.

Saxagliptin is also known as (1S,3S,5S)-2-[(2S)-2-amino-2-(3-hydroxy-1-adamantypacetyl]-2-azabicyclo[3.1.0]hexane-3-carbonitrile.

Alogliptin is also known as 2-[[6-[(3R)-3-aminopiperidin-1-yl]-3-methyl-2,4-dioxopyrimidin-1-yl]methyl]benzonitrile; benzoic acid.

Sitagliptin is also known as (3R)-3-amino-1-[3-(trifluoromethyl)-6,8-dihydro-5H-[1,2,4]triazolo[4,3-a]pyrazin-7-yl]-4-(2,4,5-trifluorophenyl)butan-1-one

Vildagliptin is also known as (2S)-1-[2-[(3-hydroxy-1-adamantyl)amino]acetyl]pyrrolidine-2-carbonitrile.

Linagliptin is also known as 8-[(3R)-3-aminopiperidin-1-yl]-7-but-2-ynyl-3-methyl-1-[(4-methylquinazolin-2-yl)methyl]purine-2,6-dione.

Thus, a preferred embodiment of the present invention relates to a compound selected from the group consisting of Saxagliptin, Alogliptin, Sitagliptin, Vildagliptin, Linagliptin and Talabostat. for use in regulating follicle maturation, such as for use in regulating the primordial to primary transition of follicles.

A particular preferred embodiment relates to a compound selected from the group consisting of Saxagliptin, Alogliptin, Sitagliptin, Vildagliptin, Linagliptin and Talabostat. for use in inhibiting follicle maturation, such as for use in inhibiting the primordial to primary transition of follicles.

A further aspect of the present invention relates to a compound selected from the group consisting of Saxagliptin, Alogliptin, Sitagliptin, Vildagliptin, Linagliptin and Talabostat for use in treating, preventing or ameliorating infertility or reduced fertility.

Another aspect of the present invention relates to a compound selected from the group consisting of Saxagliptin, Alogliptin, Sitagliptin, Vildagliptin, Linagliptin and Talabostat for use in treating, preventing or ameliorating infertility or reduced fertility in a female mammal.

### Toll Like Receptor 3 as a Target

Another aspect of the present invention relates to a compound for use in regulating follicle maturation, wherein said compound can regulate the activity of toll like receptor 3 (TLR3).

Toll like receptor 3 was found to be significantly up-regulated in granulosa cells from human primordial and primary follicles (see Table 3)

When inhibition of follicle maturation is preferred, said compound is preferably an inhibitor of TLR3. Thus, in one embodiment said compound is an inhibitor of TLR3. In a preferred embodiment said compound or inhibitor of TLR3 regulates the primordial to primary transition of follicles. In a particular preferred embodiment said compound or said inhibitor of TLR3 inhibits the primordial to primary transition of follicles.

In one particular preferred embodiment, said inhibitor is Rintatolimod. Rintatolimod is also known as [(2R,3S,4R,5R)-5-(4-amino-2-oxopyrimidin-1-yl)-3,4-dihydroxyoxolan-2-yl]methyl dihydrogen phosphate;[(2R,3S,4R,5R)-3,4-dihydroxy-5-(6-oxo-3H-purin-9-yl)oxolan-2-yl]methyl dihydrogen phosphate;[(2R,3S,4R,5R)-5-(2,4-dioxopyrimidin-1-yl)-3,4-dihydroxyoxolan-2-yl]methyl dihydrogen phosphate.

Thus, a preferred embodiment of the present invention relates to Rintatolimod for use in regulating follicle maturation, such as for use in regulating the primordial to primary transition of follicles.

A particular preferred embodiment relates to Rintatolimod for use in inhibiting follicle maturation, such as for use in inhibiting the primordial to primary transition of follicles.

A further aspect of the present invention relates Rintatolimod for use in treating, preventing or ameliorating infertility or reduced fertility.

Another aspect of the present invention relates to Rintatolimod for use in treating, preventing or ameliorating infertility or reduced fertility in a female mammal.

### Gamma-Glutamyl Carboxylase as a Target

A further aspect of the present invention relates to a compound for use in regulating follicle maturation, wherein said compound can regulate the activity of gamma-glutamyl carboxylase (GGCX).

GGCX was found to be significantly up-regulated in granulosa cells from human primordial and primary follicles (see Table 3)

When inhibition of follicle maturation is preferred, said compound is preferably an inhibitor of GGCX. Thus, in one embodiment said compound is an inhibitor of GGCX. In a preferred embodiment said compound or inhibitor of GGCX regulates the primordial to primary transition of follicles. In a particular preferred embodiment said compound or said inhibitor of GGCX inhibits the primordial to primary transition of follicles.

In one particular preferred embodiment, said inhibitor is selected from the group consisting of Anisindione and Menadione. Anisindione is also known as 2-(4-methoxyphenyl)indene-1,3-dione. Menadione is also known as 2methylnaphthalene-1,4-dione.

Thus, a preferred embodiment of the present invention relates to a compound selected from the group consisting of Anisindione and Menadione for use in regulating follicle maturation, such as for use in regulating the primordial to primary transition of follicles.

A particular preferred embodiment relates to a compound selected from the group consisting of Anisindione and Menadione for use in inhibiting follicle maturation, such as for use in inhibiting the primordial to primary transition of follicles.

A further aspect of the present invention relates to a compound selected from the group consisting of Anisindione and Menadione for use in treating, preventing or ameliorating infertility or reduced fertility.

Preferably, the invention relates to a compound selected from the group consisting of Anisindione and Menadione for use in treating, preventing or ameliorating infertility or reduced fertility in a female mammal.

### Glutamate Metabotropic Receptor 8 as a Target

A further aspect of the present invention relates to a compound for use in regulating follicle maturation, wherein said compound can regulate the activity of glutamate metabotropic receptor 8 (GRM8).

GRM8 was found to be significantly up-regulated in granulosa cells from human primordial and primary follicles (see Table 3)

When stimulation or activation of follicle maturation is preferred, said compound is preferably an activator of GRM8. Thus, in one embodiment said compound is an activator of GRM8. In a preferred embodiment said compound or activator of GRM8 regulates the primordial to primary transition of follicles. In a particular preferred embodiment said compound or said activator of GRM8 stimulates or activates the primordial to primary transition of follicles. Thus, preferably, said activator of GRM8 stimulates or activates follicle maturation.

In one particular preferred embodiment, said activator is Fasoracetam. Fasoracetam is also known as (5R)-5-(piperidine-1-carbonyl)pyrrolidin-2-one.

Thus, a preferred embodiment of the present invention relates to Fasoracetam use in regulating follicle maturation, such as for use in regulating the primordial to primary transition of follicles.

A particular preferred embodiment relates to Fasoracetam for use in stimulating or activating follicle maturation, such as for use in stimulating or activating the primordial to primary transition of follicles.

A further aspect of the present invention relates to Fasoracetam for use in treating, preventing or ameliorating infertility or reduced fertility.

Preferably, the invention relates to Fasoracetam for use in treating, preventing or ameliorating infertility or reduced fertility in a female mammal.

### Smoothened Frizzled Class Receptor as a Target

A further aspect of the present invention relates to a compound for use in regulating follicle maturation, wherein said compound can regulate the activity of smoothened, frizzled class receptor (SMO).

SMO was found to be significantly up-regulated in granulosa cells from human primordial and primary follicles (see Table 3)

When inhibition of follicle maturation is preferred, said compound is preferably an inhibitor of SMO. Thus, in one embodiment said compound is an inhibitor of SMO. In a preferred embodiment said compound or inhibitor of SMO regulates the primordial to primary transition of follicles. In a particular preferred embodiment said compound or said inhibitor of SMO inhibits the primordial to primary transition of follicles.

In one particular preferred embodiment, said inhibitor is selected from the group consisting of NVP-LEQ-506, TAK-441, PF-04449913, Taladegib, Sonidegib, Saridegib and Vismodegib.

NVP-LEQ-506 is also known as (2-[5-[(2R)-4-(6-benzyl-4,5-dimethylpyridazin-3-yl)-2-methylpiperazin-1-yl]pyrazin-2-yl]propan-2-01).

TAK-441 is also known as (6-ethyl-N-[1-(2-hydroxyacetyl)piperidin-4-yl]-1-methyl-4-oxo-5-phenacyl-3-(2,2,2-trifluoroethoxy)pyrrolo[3,2-c]pyridine-2-carboxamide). PF-04449913 is also known as 1-[(2R,4R)-2-(1H-benzimidazol-2-yl)-1-methylpiperidin-4-yl]-3-(4-cyanophenyl)urea.

Taladegib is also known as 4-fluoro-N-methyl-N-[1-[4-(2-methylpyrazol-3-yl)phthalazin-1-yl]piperidin-4-yl]-2-(trifluoromethyl)benzamide.

Sonidegib is also known as (N-[6-[(2S,6R)-2,6-dimethylmorpholin-4-yl]pyridin-3-yl]-2-methyl-3-[4-(trifluoromethoxy)phenyl]benzamide).

Saridegib is also known as N-[(3R,3′R,3′aS,4aR,6'S,6aR,6bS,7′aR,9S,12aS,12bS)-3′,6′,11,12b-tetramethylspiro[1,2,3,4,4a,5,6,6a,6b,7,8,10,12,12a-tetradecahydronaphtho[2,1-a]azulene-9,2′-3a,4,5,6,7,7a-hexahydro-3H-furo[3,2-b]pyridine]-3-yl]methanesulfonamide.

Vismodegib is also known as (2-chloro-N-(4-chloro-3-pyridin-2-ylphenyl)-4-methylsulfonylbenzamide).

Thus, a preferred embodiment of the present invention relates to a compound selected from the group consisting of NVP-LEQ-506, TAK-441, PF-04449913, Taladegib, Sonidegib, Saridegib and Vismodegib for use in regulating follicle maturation, such as for use in regulating the primordial to primary transition of follicles.

A particular preferred embodiment relates to a compound selected from the group consisting of NVP-LEQ-506, TAK-441, PF-04449913, Taladegib, Sonidegib, Saridegib and Vismodegib for use in inhibiting follicle maturation, such as for use in inhibiting the primordial to primary transition of follicles.

A further aspect of the present invention relates to a compound selected from the group consisting of NVP-LEQ-506, TAK-441, PF-04449913, Taladegib, Sonidegib, Saridegib and Vismodegib for use in treating, preventing or ameliorating infertility or reduced fertility.

Preferably, the invention relates to a compound selected from the group consisting of NVP-LEQ-506, TAK-441, PF-04449913, Taladegib, Sonidegib, Saridegib and Vismodegib for use in treating, preventing or ameliorating infertility or reduced fertility in a female mammal.

### Potassium Voltage-Gated Channel Subfamily B Member 1 as a Target

Another aspect of the present invention relates to a compound for use in regulating follicle maturation, wherein said compound can regulate the activity of potassium voltage-gated channel subfamily B member 1 (KCNB1).

KCNB1 was found to be significantly down-regulated in granulosa cells from human primordial and primary follicles (see Table 3)

When stimulation or activation of follicle maturation is preferred, said compound is preferably an inhibitor of KCNB1. Thus, in one embodiment said compound is an inhibitor of KCNB1. In a preferred embodiment said compound or inhibitor of KCNB1 regulates the primordial to primary transition of follicles. In a particular preferred embodiment said compound or said inhibitor of KCNB1 stimulates the primordial to primary transition of follicles. Thus, preferably, said inhibitor of KCNB1 stimulates or activates follicle maturation.

In one particular preferred embodiment, said inhibitor is Dalfampridine.

Thus, a preferred embodiment of the present invention relates to Dalfampridine use in regulating follicle maturation, such as for use in regulating the primordial to primary transition of follicles.

A particular preferred embodiment relates to Dalfampridine for use in stimulating or activating follicle maturation, such as for use in stimulating or activating the primordial to primary transition of follicles.

A further aspect of the present invention relates to Dalfampridine for use in treating, preventing or ameliorating infertility or reduced fertility.

Preferably, the invention relates to Dalfampridine for use in treating, preventing or ameliorating infertility or reduced fertility in a female mammal.

### Prolactin Receptor as a Target

A further aspect of the present invention relates to a compound for use in regulating follicle maturation, wherein said compound can regulate the activity of Prolactin receptor (PRLR).

PRLR was found to be significantly down-regulated in granulosa cells from human primordial and primary follicles (see Table 3)

When inhibition of follicle maturation is preferred, said compound is preferably an inhibitor of PRLR. Thus, in one embodiment said compound is an inhibitor of PRLR. In a preferred embodiment said compound or inhibitor of PRLR regulates the primordial to primary transition of follicles. In a particular preferred embodiment said compound or said inhibitor of PRLR inhibits the primordial to primary transition of follicles. Thus, preferably PRLR inhibits follicle maturation.

In one particular preferred embodiment, said inhibitor is Fluoxymesterone. Fluoxymesterone is also known as (8S,9R,10S,11S,13S,14S,17S)-9-fluoro-11,17-dihydroxy-10,13,17-trimethyl-1,2,6,7,8,11,12,14,15,16-decahydrocyclopenta[a]phenanthren-3-one.

Thus, a preferred embodiment of the present invention relates to Fluoxymesterone for use in regulating follicle maturation, such as for use in regulating the primordial to primary transition of follicles.

A particular preferred embodiment relates to Fluoxymesterone for use in inhibiting follicle maturation, such as for use in inhibiting the primordial to primary transition of follicles.

A further aspect of the present invention relates to Fluoxymesterone for use in treating, preventing or ameliorating infertility or reduced fertility.

Preferably, the invention relates to Fluoxymesterone for use in treating, preventing or ameliorating infertility or reduced fertility in a female mammal.

### Cyclin Dependent Kinase 1 as a Target

Another aspect of the present invention relates to a compound for use in regulating follicle maturation, wherein said compound can regulate the activity of cyclin dependent kinase 1 (CDK1).

CDK1 was found to be significantly down-regulated in granulosa cells from human primordial and primary follicles (see Table 3)

When stimulation or activation of follicle maturation is preferred, said compound is preferably an inhibitor of CDK1. Thus, in one embodiment said compound is an inhibitor of CDK1. In a preferred embodiment said compound or inhibitor of CDK1 regulates the primordial to primary transition of follicles. In a particular preferred embodiment said compound or said inhibitor of CDK1 stimulates the primordial to primary transition of follicles. Thus, preferably, said inhibitor of CDK1 stimulates or activates follicle maturation.

In one particular preferred embodiment, said inhibitor is selected from the group consisting of Dinaciclib, Milciclib, Roniciclib and Alvocidib.

Dinaciclib is also known as 2-[(2S)-1-[3-ethyl-7-[(1-oxidopyridin-1-ium-3-yl)methylamino]pyrazolo[1,5-a]pyrimidin-5-yl]piperidin-2-yl]ethanol).

Milciclib is also known as N,1,4,4-tetramethyl-8[4-(4-methylpiperazin-1-yl)anilino]-5H-pyrazolo[4,3-h]quinazoline-3-carboxamide.

Roniciclib is also known as (2R,3R)-3-[2-[4-(cyclopropylsulfonimidoyl)anilino]-5-(trifluoromethyl)pyrimidin-4-yl]oxybutan-2-ol.

Alvocidib is also known as 2-(2-chlorophenyl)-5,7-dihydroxy-8-[(3S,4R)-3-hydroxy-1-methylpiperidin-4-yl]chromen-4-one.

Thus, a preferred embodiment of the present invention relates an inhibitor from the group consisting of Dinaciclib, Milciclib, Roniciclib and Alvocidib for use in regulating follicle maturation, such as for use in regulating the primordial to primary transition of follicles.

A particular preferred embodiment relates to a compound from the group consisting of Dinaciclib, Milciclib, Roniciclib and Alvocidib for use in stimulating or activating follicle maturation, such as for use in stimulating or activating the primordial to primary transition of follicles.

A further aspect of the present invention relates to a compound from the group consisting of Dinaciclib, Milciclib, Roniciclib and Alvocidib for use in treating, preventing or ameliorating infertility or reduced fertility.

Preferably, the invention relates to a compound from the group consisting of Dinaciclib, Milciclib, Roniciclib and Alvocidib for use in treating, preventing or ameliorating infertility or reduced fertility in a female mammal.

Neurotrophic receptor tyrosine kinase 2 as a target A further aspect of the present invention relates to a compound for use in regulating follicle maturation, wherein said compound can regulate the activity neurotrophic receptor tyrosine kinase 2 (NTRK2).

NTRK2 was found to be significantly down-regulated in granulosa cells from human primordial and primary follicles (see Table 3)

When inhibition of follicle maturation is preferred, said compound is preferably an inhibitor of NTRK2. Thus, in one embodiment said compound is an inhibitor of NTRK2. In a preferred embodiment said compound or inhibitor of NTRK2 regulates the primordial to primary transition of follicles. In a particular preferred embodiment said compound or said inhibitor of NTRK2 inhibits the primordial to primary transition of follicles. Thus, preferably NTRK2 inhibits follicle maturation.

In one particular preferred embodiment, said inhibitor is selected from the group consisting of Cabozantinib, Theophylline and Entrectinib.

Thus, a preferred embodiment of the present invention relates to a compound selected from the group consisting of Cabozantinib, Theophylline and Entrectinib for use in regulating follicle maturation, such as for use in regulating the primordial to primary transition of follicles. Cabozantinib is also known as 1-N-[4-(6,7-dimethoxyquinolin-4-yl)oxyphenyl]-1-N′-(4-fluorophenyl)cyclopropane-1,1-dicarboxamide. Theophylline is a methyl xanthine derivative also known as 1,3-dimethyl-7H-purine-2,6-dione. Entrectinib is also known as N-[5-[(3,5-difluorophenyl)methyl]-1H-indazol-3-yl]-4-(4-methylpiperazin-1-yl)-2-(oxan-4-ylamino)benzamide.

A particular preferred embodiment relates to a compound selected from the group consisting of Cabozantinib, Theophylline and Entrectinib for use in inhibiting follicle maturation, such as for use in inhibiting the primordial to primary transition of follicles.

A further aspect of the present invention relates to a compound selected from the group consisting of Cabozantinib, Theophylline and Entrectinib for use in treating, preventing or ameliorating infertility or reduced fertility.

Preferably, the invention relates to a compound selected from the group consisting of Cabozantinib, Theophylline and Entrectinib for use in treating, preventing or ameliorating infertility or reduced fertility in a female mammal.

### Phospholipase C Like 1 as a Target

A further aspect of the present invention relates to a compound for use in regulating follicle maturation, wherein said compound can regulate the activity of phospholipase C like 1 (PLCL1).

PLCL1 was found to be significantly down-regulated in granulosa cells from human primordial and primary follicles (see Table 3)

When inhibition of follicle maturation is preferred, said compound is preferably an inhibitor of PLCL1. Thus, in one embodiment said compound is an inhibitor of PLCL1. In a preferred embodiment said compound or inhibitor of PLCL1 regulates the primordial to primary transition of follicles. In a particular preferred embodiment said compound or said inhibitor of PLCL1 inhibits the primordial to primary transition of follicles. Thus, preferably PLCL1 inhibits follicle maturation.

In one particular preferred embodiment, said inhibitor is Quinacrine. Quinacrine is also known as 4-N-(6-chloro-2-methoxyacridin-9-yl)-1-N,1-N-diethylpentane-1,4-diamine.

Thus, a preferred embodiment of the present invention relates to Quinacrine for use in regulating follicle maturation, such as for use in regulating the primordial to primary transition of follicles.

A particular preferred embodiment relates to Quinacrine for use in inhibiting follicle maturation, such as for use in inhibiting the primordial to primary transition of follicles.

A further aspect of the present invention relates to Quinacrine for use in treating, preventing or ameliorating infertility or reduced fertility.

Preferably, the invention relates to Quinacrine for use in treating, preventing or ameliorating infertility or reduced fertility in a female mammal.

### Gamma-Aminobutyric Acid Type A Receptor Epsilon Subunit as a Target

A further aspect of the present invention relates to a compound for use in regulating follicle maturation, wherein said compound can regulate the activity of gamma-aminobutyric acid type A receptor epsilon subunit (GABRE).

GABRE was found to be significantly down-regulated in granulosa cells from human primordial and primary follicles (see Table 3)

When stimulation or activation of follicle maturation is preferred, said compound is preferably an activator of GABRE. Thus, in one embodiment said compound is an activator of GABRE. In a preferred embodiment said compound or activator of GABRE regulates the primordial to primary transition of follicles. In a particular preferred embodiment said compound or said activator of GABRE stimulates or activates the primordial to primary transition of follicles. Thus, preferably, said activator of GABRE stimulates or activates follicle maturation.

In one particular preferred embodiment, said activator is selected from the group consisting of Eszopiclone and Pregnenolone. Eszopiclone is also known as [(7S)-6-(5-chloropyridin-2-yl)-5-oxo-7H-pyrrolo[3,4-b]pyrazin-7-yl] 4-methylpiperazine-1-carboxylate. Pregnenolone is also known as 1-[(3S,8S,9S,10R,13S,14S,17S)-3-hydroxy-10,13-dimethyl-2,3,4,7,8,9,11,12,14,15,16,17-dodecahydro-1H-cyclopenta[a]phenanthren-17-yl]ethanone.

Thus, a preferred embodiment of the present invention relates to a compound selected from the group consisting of Eszopiclone and Pregnenolone for use in regulating follicle maturation, such as for use in regulating the primordial to primary transition of follicles.

A particular preferred embodiment relates to a compound selected from the group consisting of Eszopiclone and Pregnenolone for use in stimulating or activating follicle maturation, such as for use in stimulating or activating the primordial to primary transition of follicles.

A further aspect of the present invention relates to a compound selected from the group consisting of Eszopiclone and Pregnenolone for use in treating, preventing or ameliorating infertility or reduced fertility.

Preferably, the invention relates to a compound selected from the group consisting of Eszopiclone and Pregnenolone for use in treating, preventing or ameliorating infertility or reduced fertility in a female mammal.

### Protein Tyrosine Phosphatase Non-Receptor Type 1 as a Target

A further aspect of the present invention relates to a compound for use in regulating follicle maturation, wherein said compound can regulate the activity of protein tyrosine phosphatase non-receptor type 1 (PTPN1).

PTPN1 was found to be significantly down-regulated in granulosa cells from human primordial and primary follicles (see Table 3)

When inhibition of follicle maturation is preferred, said compound is preferably an inhibitor of PTPN1. Thus, in one embodiment said compound is an inhibitor of PTPN1. In a preferred embodiment said compound or inhibitor of PTPN1 regulates the primordial to primary transition of follicles. In a particular preferred embodiment said compound or said inhibitor of PTPN1 inhibits the primordial to primary transition of follicles. Thus, preferably PTPN1 inhibits follicle maturation.

In one particular preferred embodiment, said inhibitor is Trodusquemine. Trodusquemine is also known as [(3R,6R)-6-[(3S,5R,7R,8R,9S,10S,13R,14S,17R)-3-[3-[4-(3-aminopropylamino)butylamino]propylamino]-7-hydroxy-10,13-dimethyl-2,3,4,5,6,7,8,9,11,12,14,15,16,17-tetradecahydro-1H-cyclopenta[a]phenanthren-17-yl]-2-methylheptan-3-yl] hydrogen sulfate.

Thus, a preferred embodiment of the present invention relates to Trodusquemine for use in regulating follicle maturation, such as for use in regulating the primordial to primary transition of follicles.

A particular preferred embodiment relates to Trodusquemine for use in inhibiting follicle maturation, such as for use in inhibiting the primordial to primary transition of follicles.

A further aspect of the present invention relates to Trodusquemine for use in treating, preventing or ameliorating infertility or reduced fertility.

Preferably, the invention relates to Trodusquemine for use in treating, preventing or ameliorating infertility or reduced fertility in a female mammal.

### Xanthine Dehydrogenase as a Target

A further aspect of the present invention relates to a compound for use in regulating follicle maturation, wherein said compound can regulate the activity of xanthine dehydrogenase (XDH).

XDH was found to be significantly down-regulated in granulosa cells from human primordial and primary follicles (see Table 3)

When inhibition of follicle maturation is preferred, said compound is preferably an inhibitor of XDH. Thus, in one embodiment said compound is an inhibitor of XDH. In a preferred embodiment said compound or inhibitor of XDH regulates the primordial to primary transition of follicles. In a particular preferred embodiment said compound or said inhibitor of XDH inhibits the primordial to primary transition of follicles. Thus, preferably XDH inhibits follicle maturation.

In one particular preferred embodiment, said inhibitor is selected from the group consisting of Allopurinol, Oxypurinol and Febuxostat.

Allopurinol is also known as 1,2-dihydropyrazolo[3,4-d]pyrimidin-4-one. Oxypurinol is also known as 1,2-dihydropyrazolo[3,4-d]pyrimidine-4,6-dione. Febuxostat is also known as 2-[3-cyano-4-(2-methylpropoxy)phenyl]-4-methyl-1,3-thiazole-5-carboxylic acid.

Thus, a preferred embodiment of the present invention relates to a compound selected from the group consisting of Allopurinol, Oxypurinol and Febuxostat for use in regulating follicle maturation, such as for use in regulating the primordial to primary transition of follicles.

A particular preferred embodiment relates to a compound selected from the group consisting of Allopurinol, Oxypurinol and Febuxostat for use in inhibiting follicle maturation, such as for use in inhibiting the primordial to primary transition of follicles.

A further aspect of the present invention relates to a compound selected from the group consisting of Allopurinol, Oxypurinol and Febuxostat for use in treating, preventing or ameliorating infertility or reduced fertility.

Preferably, the invention relates to a compound selected from the group consisting of Allopurinol, Oxypurinol and Febuxostat for use in treating, preventing or ameliorating infertility or reduced fertility in a female mammal.

### WEE1 G2 Checkpoint Kinase as a Target

A further aspect of the present invention relates to a compound for use in regulating follicle maturation, wherein said compound can regulate the activity of WEE1 G2 checkpoint kinase (WEE1).

WEE1 was found to be significantly down-regulated in granulosa cells from human primordial and primary follicles (see Table 3)

When inhibition of follicle maturation is preferred, said compound is preferably an inhibitor of WEE1. Thus, in one embodiment said compound is an inhibitor of WEE1. In a preferred embodiment said compound or inhibitor of WEE1 regulates the primordial to primary transition of follicles. In a particular preferred embodiment said compound or said inhibitor of WEE1 inhibits the primordial to primary transition of follicles. Thus, preferably WEE1 inhibits follicle maturation.

In one particular preferred embodiment, said inhibitor is MK 1775. MK 1775 is also known as 1-[6-(2-hydroxypropan-2-yl)pyridin-2-yl]-6-[4-(4-methylpiperazin-1-yl)anilino]-2-prop-2-enylpyrazolo[3,4-d]pyrimidin-3-one.

Thus, a preferred embodiment of the present invention relates to MK 1775 for use in regulating follicle maturation, such as for use in regulating the primordial to primary transition of follicles.

A particular preferred embodiment relates to MK 1775 for use in inhibiting follicle maturation, such as for use in inhibiting the primordial to primary transition of follicles.

A further aspect of the present invention relates to MK 1775 for use in treating, preventing or ameliorating infertility or reduced fertility.

Preferably, the invention relates to MK 1775 for use in treating, preventing or ameliorating infertility or reduced fertility in a female mammal.

### Protein Kinase C Beta as a Target

A further aspect of the present invention relates to a compound for use in regulating follicle maturation, wherein said compound can regulate the activity of protein kinase C beta (PRKCB).

PRKCB was found to be significantly down-regulated in granulosa cells from human primordial and primary follicles (see Table 3)

When inhibition of follicle maturation is preferred, said compound is preferably an inhibitor of PRKCB. Thus, in one embodiment said compound is an inhibitor of PRKCB. In a preferred embodiment said compound or inhibitor of PRKCB regulates the primordial to primary transition of follicles. In a particular preferred embodiment said compound or said inhibitor of PRKCB inhibits the primordial to primary transition of follicles. Thus, preferably PRKCB inhibits follicle maturation.

In one particular preferred embodiment, said inhibitor is selected from the group consisting of Sotrastaurin, Enzastaurin and Ruboxistaurin.

Sotrastaurin is also known as 3-(1H-indol-3-yl)-4-[2-(4-methylpiperazin-1-yl)quinazolin-4-yl]pyrrole-2,5-dione.

Enzastaurin is also known as 3-(1-methylindol-3-yl)-4-[1-[1-(pyridin-2-ylmethyl)piperidin-4-yl]indol-3-yl]pyrrole-2,5-dione.

Ruboxistaurin is also known as 13-((dimethylamino)methyl)-10,11,14,15-tetrahydro-4,9:16,21-dimetheno-1H,13H-dibenzo(e,k)pyrrolo(3,4-h)(1,4,13)oxadiazacyclohexadecene-1,3(2H)-dione.

Thus, a preferred embodiment of the present invention relates to a compound selected from the group consisting of Sotrastaurin, Enzastaurin and Ruboxistaurin for use in regulating follicle maturation, such as for use in regulating the primordial to primary transition of follicles.

A particular preferred embodiment relates to a compound selected from the group consisting of Sotrastaurin, Enzastaurin and Ruboxistaurin for use in inhibiting follicle maturation, such as for use in inhibiting the primordial to primary transition of follicles.

A further aspect of the present invention relates to a compound selected from the group consisting of Sotrastaurin, Enzastaurin and Ruboxistaurin for use in treating, preventing or ameliorating infertility or reduced fertility.

Preferably, the invention relates to a compound selected from the group consisting of Sotrastaurin, Enzastaurin and Ruboxistaurin for use in treating, preventing or ameliorating infertility or reduced fertility in a female mammal.

### Arachidonate 12-Lipoxygenase 12S Type as a Target

A further aspect of the present invention relates to a compound for use in regulating follicle maturation, wherein said compound can regulate the activity of arachidonate 12-lipoxygenase 12S type (ALOX12).

ALOX12 was found to be significantly down-regulated in granulosa cells from human primordial and primary follicles (see Table 3)

When inhibition of follicle maturation is preferred, said compound is preferably an inhibitor of ALOX12. Thus, in one embodiment said compound is an inhibitor of ALOX12. In a preferred embodiment said compound or inhibitor of ALOX12 regulates the primordial to primary transition of follicles. In a particular preferred embodiment said compound or said inhibitor of ALOX12 inhibits the primordial to primary transition of follicles. Thus, preferably ALOX12 inhibits follicle maturation.

In one particular preferred embodiment, said inhibitor is selected from the group consisting of Sulfasalazine, Balsalazide, Mesalamine and Masoprocol.

Sulfasalazine is also known as (3Z)-6-oxo-3-[[4-(pyridin-2-ylsulfamoyl)phenyl]hydrazinylidene]cyclohexa-1,4-diene-1-carboxylic acid. Balsalazide is also known as (3Z)-3-[[4-(2-carboxyethylcarbamoyl)phenyl]hydrazinylidene]-6-oxocyclohexa-1,4-diene-1-carboxylic acid.

Mesalamine is also known as 5-amino-2-hydroxybenzoic acid.

Masoprocol is also known as 4-[(2S,3R)-4-(3,4-dihydroxyphenyl)-2,3-dimethylbutyl]benzene-1,2-diol.

Thus, a preferred embodiment of the present invention relates to a compound selected from the group consisting of Sulfasalazine, Balsalazide, Mesalamine and Masoprocol for use in regulating follicle maturation, such as for use in regulating the primordial to primary transition of follicles.

A particular preferred embodiment relates to a compound selected from the group consisting of Sulfasalazine, Balsalazide, Mesalamine and Masoprocol for use in inhibiting follicle maturation, such as for use in inhibiting the primordial to primary transition of follicles.

A further aspect of the present invention relates to a compound selected from the group consisting of Sulfasalazine, Balsalazide, Mesalamine and Masoprocol for use in treating, preventing or ameliorating infertility or reduced fertility.

Preferably, the invention relates to a compound selected from the group consisting of Sulfasalazine, Balsalazide, Mesalamine and Masoprocol for use in treating, preventing or ameliorating infertility or reduced fertility in a female mammal.

### Amine Oxidase Copper Containing 3 as a Target

A further aspect of the present invention relates to a compound for use in regulating follicle maturation, wherein said compound can regulate the activity of amine oxidase copper containing 3 (AOC3).

AOC3 was found to be significantly down-regulated in granulosa cells from human primordial and primary follicles (see Table 3)

When inhibition of follicle maturation is preferred, said compound is preferably an inhibitor of AOC3. Thus, in one embodiment said compound is an inhibitor of AOC3. In a preferred embodiment said compound or inhibitor of AOC3 regulates the primordial to primary transition of follicles. In a particular preferred embodiment said compound or said inhibitor of AOC3 inhibits the primordial to primary transition of follicles. Thus, preferably AOC3 inhibits follicle maturation.

In one particular preferred embodiment, said inhibitor is Hydralazine. Hydralazine is also known as phthalazin-1-ylhydrazine.

Thus, a preferred embodiment of the present invention relates to Hydralazine for use in regulating follicle maturation, such as for use in regulating the primordial to primary transition of follicles.

A particular preferred embodiment relates to Hydralazine for use in inhibiting follicle maturation, such as for use in inhibiting the primordial to primary transition of follicles.

A further aspect of the present invention relates to Hydralazine for use in treating, preventing or ameliorating infertility or reduced fertility.

Preferably, the invention relates Hydralazine for use in treating, preventing or ameliorating infertility or reduced fertility in a female mammal.

### Solute Carrier Family 52 Member 2 as a Target

A further aspect of the present invention relates to a compound for use in regulating follicle maturation, wherein said compound can regulate the activity of solute carrier family 52 member 2 (SLC52A2).

SLC52A2 was found to be significantly down-regulated in granulosa cells from human primordial and primary follicles (see Table 3)

When inhibition of follicle maturation is preferred, said compound is preferably an inhibitor of SLC52A2. Thus, in one embodiment said compound is an inhibitor of SLC52A2. In a preferred embodiment said compound or inhibitor of SLC52A2 regulates the primordial to primary transition of follicles. In a particular preferred embodiment said compound or said inhibitor of SLC52A2 inhibits the primordial to primary transition of follicles. Thus, preferably SLC52A2 inhibits follicle maturation.

In one particular preferred embodiment, said inhibitor is 4-hydroxybutanoic acid. 4-hydroxybutanoic acid is also known as 4-Amino-3-hydroxybutyrate.

Thus, a preferred embodiment of the present invention relates to 4-hydroxybutanoic acid for use in regulating follicle maturation, such as for use in regulating the primordial to primary transition of follicles.

A particular preferred embodiment relates to 4-hydroxybutanoic acid for use in inhibiting follicle maturation, such as for use in inhibiting the primordial to primary transition of follicles.

A further aspect of the present invention relates to 4-hydroxybutanoic acid for use in treating, preventing or ameliorating infertility or reduced fertility.

Preferably, the invention relates 4-hydroxybutanoic acid for use in treating, preventing or ameliorating infertility or reduced fertility in a female mammal.

### Use of the Compounds According to the Present Invention

The compounds according to the present invention can be used in vivo wherein they are administered to a female individual in need thereof. Thus, in one embodiment the compounds as described herein and above are for use in regulating follicle maturation in vivo.

The compounds can also be used in vitro wherein they are added in vitro to follicles. Thus, in another embodiment the compounds as described herein and above are for use in regulating follicle maturation in vitro.

In a preferred embodiment of the present invention the compounds as described herein and above are for use in regulating primordial to primary transition of follicles. Thus, an aspect of the present invention relates to a compound as defined herein and above for use in regulating primordial to primary transition of follicles.

In a preferred embodiment said follicles are mammalian follicles. The mammalian can be any mammal such as for example a rodent, a primate, or a carnivore. In a preferred embodiment the rodent is a mouse.

In a preferred embodiment, the mammalian is a human.

The compounds as defined herein and above can be used to exert pharmacological control of follicle regulation, in particular primordial to primary follicle regulation.

Several diseases or disorders are associated with poor control in the first step of follicle regulation, namely primordial to primary follicle regulation. These disorders are commonly referred to as ovulation disorders. Problems with the regulation of reproductive hormones by the hypothalamus or the pituitary gland, or problems in the ovary, can cause ovulation disorders.

Thus, a further aspect of the present invention relates to a compound as described and defined herein and above for use in treating, preventing or ameliorating infertility or reduced fertility.

For example, the present invention relates to a compound as described and defined herein and above for use in treating, preventing or ameliorating infertility or reduced fertility in a female individual.

Preferably said female individual is a female mammal. The mammal is as defined above. Thus, preferably, said female mammal is a female human.

An aspect of the present invention relates to a compound as described and defined herein and above for use in treating, preventing or ameliorating an ovulation disorder, such as an ovulation disorder.

In one embodiment said ovulation disorder is an infertility disorder.

In one embodiment said ovulation disorder is selected from the group consisting of Polycystic ovary syndrome (PCOS), Premature ovarian failure (P01), Hypothalamic dysfunction and Menopause.

PCOS causes a hormone imbalance, which affects ovulation (amoungst other, it an endoctien metabolic disease). PCOS is a condition that causes women to not ovulate, or to ovulate irregularly. PCOS is associated with insulin resistance and obesity, abnormal hair growth on the face or body, and acne. PCOS is the most common cause of female infertility.

Premature ovarian failure (P01), also called primary ovarian insufficiency, is a disorder usually caused by an autoimmune response or by premature loss of eggs from your ovary (possibly from genetics or chemotherapy). POI, sometimes referred to as premature menopause, occurs when a woman's ovaries fail before she is 40 years of age The ovary no longer produces eggs, and it lowers estrogen production in women under the age of 40. Although certain exposures, such as chemotherapy or pelvic radiation therapy, and certain medical conditions may cause POI, the cause is often unexplained.

Hypothalamic dysfunction also called Functional hypothalamic amenorrhea (FHA) is a condition caused by excessive exercise, stress, or low body weight. It is sometimes associated with eating disorders such as anorexia. Two hormones produced by the pituitary gland are responsible for stimulating ovulation each month—(FSH) and luteinizing hormone (LH). Excess physical or emotional stress, a very high or very low body weight, or a recent substantial weight gain or loss can disrupt production of these hormones and affect ovulation. Irregular or absent periods are the most common signs.

The ovulation disorder may also be caused by hyperprolactinemia, wherein too much prolactin is produced. For example, the pituitary gland may cause excess production of prolactin (hyperprolactinemia), which reduces estrogen production and may cause infertility. Usually related to a pituitary gland problem, this can also be caused by medications you're taking for another disease.

In another embodiment the ovulation disorder is caused by improper function of the hypothalamus and pituitary glands. The hypothalamus and pituitary glands in the brain produce hormones that maintain normal ovarian function. Production of too much of the hormone prolactin by the pituitary gland (often as the result of a benign pituitary gland tumor), or improper function of the hypothalamus or pituitary gland, may cause a woman not to ovulate.

### Pharmaceutical Formulation

Whilst it is possible for the compounds of the present invention to be administered alone, it is preferred to present them in the form of a pharmaceutical formulation.

Thus, another aspect of the present invention relates to a pharmaceutical composition comprising at least one compound as defined herein for use in treating, preventing or ameliorating ameliorating infertility or reduced fertility.

For example, the present invention relates to a pharmaceutical composition comprising at least one compound as defined herein for use in treating, preventing or ameliorating infertility or reduced fertility in a female mammal.

Preferably said female individual is a female mammal. The mammal is as defined above. Thus, preferably, said female mammal is a female human.

An aspect of the present invention relates to a pharmaceutical composition comprising at least one compound as defined herein for use in treating, preventing or ameliorating an ovulation disorder, such as an infertility disorder.

The ovulation disorder is as described herein and above.

It is preferred that the pharmaceutical composition further comprises at least one pharmaceutically acceptable carrier. Suitable carriers and the formulation of such pharmaceuticals are known to a person skilled in the art.

The pharmaceutically acceptable carriers can be either solid or liquid. Solid form preparations include powders, tablets, pills, capsules, cachets, suppositories, and dispersible granules. A solid carrier can be one or more excipients which may also act as diluents, flavoring agents, solubilizers, lubricants, suspending agents, binders, preservatives, wetting agents, tablet disintegrating agents, or an encapsulating material.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The compounds of the present invention may be formulated for parenteral administration and may be presented in unit dose form in ampoules, pre-filled syringes, small volume infusion or in multi-dose containers, optionally with an added preservative. The compositions may take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils (e.g., olive oil), and injectable organic esters (e.g., ethyl oleate), and may contain agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

Preferably, the formulation will comprise about 0.5% to 75% by weight of the active ingredient(s) with the remainder consisting of suitable pharmaceutical excipients as described herein.

Pharmaceutically acceptable salts of the instant compounds, where they can be prepared, are also intended to be covered by this invention. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

The compounds of the present invention may be formulated in a wide variety of formulations for parenteral administration.

For injections and infusions the formulations may take such forms as suspensions, solutions, or emulsions in oily or aqueous vehicles, for example solutions in aqueous polyethylene glycol. Alternatively, the active ingredient may be in powder form, obtained by aseptic isolation of sterile solid or by lyophilisation from solution for constitution before use with a suitable vehicle, e.g., sterile, pyrogen-free water. The formulations can be presented in unit-dose or multi-dose sealed containers, such as ampoules, vials, pre-filled syringes, infusion bags, or can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Examples of oily or non-aqueous carriers, diluents, solvents or vehicles include propylene glycol, polyethylene glycol, vegetable oils, and injectable organic esters, and may contain formulatory agents such as preserving, wetting, emulsifying or suspending, stabilizing and/or dispersing agents.

The formulations for injection will typically contain from about 0.5 to about 25% by weight of the active ingredient in solution.

The compounds of the present invention may be formulated in a wide variety of formulations for oral administration. Solid form preparations may include powders, tablets, drops, capsules, cachets, lozenges, and dispersible granules. Other forms suitable for oral administration may include liquid form preparations including emulsions, syrups, elixirs, aqueous solutions, aqueous suspensions, or solid form preparations which are intended to be converted shortly before use to liquid form preparations, such as solutions, suspensions, and emulsions.

In powders, the carrier is a finely divided solid which is a mixture with the finely divided active component. In tablets, the active component is mixed with the carrier having the necessary binding capacity in suitable proportions and compacted in the shape and size desired. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. Suitable solvents for the preparation of an oily solution include glycerol, diluted alcohol and propylene glycol.

Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavors, stabilizing and thickening agents. Aqueous suspensions can be prepared by dispersing the finely divided active component in water with viscous material, such as natural or synthetic gums, resins, methylcellulose, sodium carboxymethylcellulose, and other well-known suspending agents.

In one embodiment the pharmaceutical composition comprises an additional active agent. The pharmaceutical composition may also comprise a combination of the compounds as described herein and above.

### Administration Forms

As described herein above administration forms include but are not limited to oral, parental, enteral, rectal or buccal administration.

In one embodiment the pharmaceutical composition is administered or adapted for administration enterally, parenterally or as part of a sustained release implant. The parenteral administration may for example be intravenous, subcutaneous, intramuscular, intracranial or intraperitoneal.

In a preferred embodiment the pharmaceutical composition is administered by or adapted for injection. In a preferred embodiment the pharmaceutical composition is administered by or adapted for injection into the ovaries.

It is appreciated that the pharmaceutical composition of the present invention comprises at least 30 wt. % compound, such as at least 25 wt. % compound, such as for example at least 20 wt. % compound, at least 15 wt. % compound, such as at least 25 wt. % compound, such as for example at least 20 wt. % compound, at least 15 wt. % compound, such as at least 10 wt. % compound, such as for example at least 8 wt. % compound, at least 5 wt. % compound, such as at least 4 wt. % compound, such as for example at least 3 wt. % compound, at least 2 wt. % compound, such as at least 1 wt. % compound, such as for example at least 0.5 wt. % compound or at least 0.5 wt. % compound.

Wt. % is an abbreviation for weight percent. The compound is any compound defined herein.

In one embodiment the compound as described herein is to be administered in a dosage of from 1 μg/kg-30,000 μg/kg body weight, such as 1 μg/kg-7,500 μg/kg, such as 1 μg/kg-5,000 μg/kg, such as 1 μg/kg-2,000 μg/kg, such as 1 μg/kg-1,000 μg/kg, such as 1 μg/kg-700 μg/kg, such as 5 μg/kg-500 μg/kg, such as 10 μg/kg to 100 μg/kg bodyweight. In another embodiment the compound as described herein is to be administered in a dosage of from 1 μg/kg-1,000 μg/kg body weight, such as 1 μg/kg-500 μg/kg, such as 1 μg/kg-250 μg/kg, such as 1 μg/kg-100 μg/kg, such as 1 μg/kg-50 μg/kg, such as 1 μg/kg to 10 μg/kg bodyweight. In yet another embodiment the compound as described herein is to be administered in a dosage of from 10 μg/kg-30,000 μg/kg body weight, such as 10 μg/kg-7,500 μg/kg, such as 10 μg/kg-5,000 μg/kg, such as 10 μg/kg-2,000 μg/kg, such as 10 μg/kg-1,000 μg/kg, such as 10 μg/kg-700 μg/kg, such as 10 μg/kg-500 μg/kg, such as 10 μg/kg to 100 μg/kg bodyweight.

In one embodiment the administration of the pharmaceutical composition as described herein is repeated at least 1, 2, 3, 4, 5 or 6 times weekly. In another embodiment the administration is repeated at least 1-3 times weekly, such as 2-5 times weekly, such as 3-6 times weekly.

The pharmaceutical composition of the present invention may also be administered in vitro to primordial follicles from said female individual. Thus, primordial follicles can be taken from an individual in need thereof, treated in vitro with at least one compound of the present invention and re-inserted into said female individual.

In one embodiment said female individual is a female mammal. Preferably, said female mammal is a female human.

### Methods

A further aspect of the present invention relates to a method for treating, preventing or ameliorating infertility of a female individual having an ovulation disorder, comprising administering to said individual a therapeutically effective amount of a compound as defined herein and above.

The compound may also be administered in the form of a pharmaceutical composition as described herein and above. Administrations forms and dosages are as described herein and above.

The ovulation disorder is as described herein and above. In one embodiment said ovulation disorder is an infertility disorder.

Preferably, said female individual is a female mammal, such as a female human.

### EXAMPLES

### Materials and Methods

### Participants

Ovarian cortical tissue was obtained from three patients undergoing oophorectomy prior to gonadotoxic treatment of a malignant disease. The primary diagnoses were unrelated to ovarian malignancies. Patients were normo-ovulatory, non-stimulated and with normal levels of reproductive hormones. In connection to oophorectomy, a small piece of the ovarian cortex is used for evaluating the ovarian reserve, and for research purposes. The ethical committee of the municipalities of Copenhagen and Frederiksberg (H-2-2011-044) approved the study and informed consent was obtained from all subjects. Ovarian cortical tissue was collected at various time points in the menstrual cycle. The ovaries were, in accordance with their morphological appearance upon excision, considered normal. Following cryopreservation, one small piece of ovarian cortex from each patient was randomly donated for the current study. Until use, the cortical piece was stored in liquid nitrogen (−196° C.), as previously described (Andersen et al. Two successful pregnancies following autotransplantation of frozen/thawed ovarian tissue, Human reproduction 2008; 23: 2266-2272; Rosendahl M et al., Cryopreservation of ovarian tissue for a decade in Denmark: a view of the technique. Reproductive biomedicine online 2011; 22: 162-171).

### Laser Capture Microdissection (LCM) and NGS

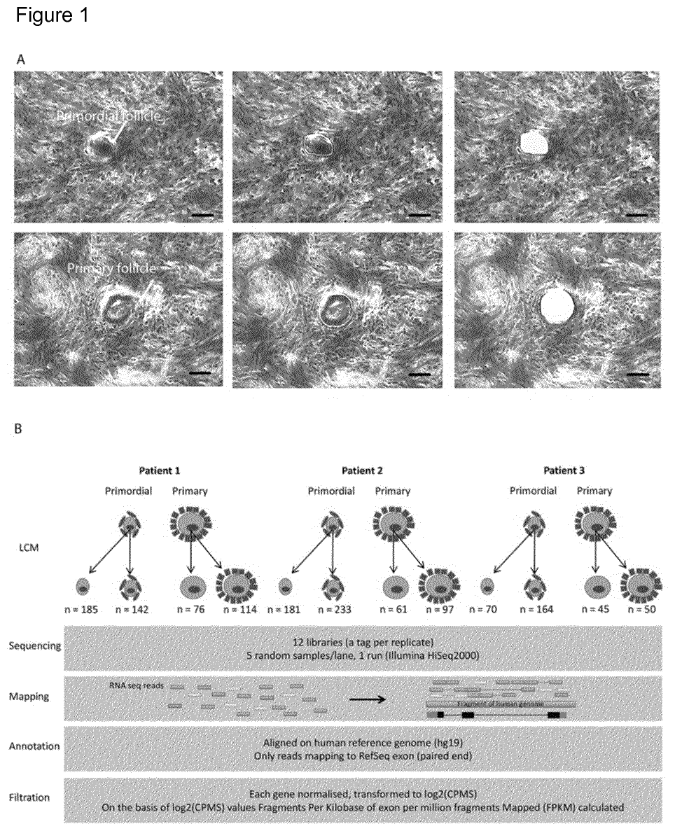

Ovarian cortical fragments with a size of 2×2×1 mm were thawed and fixed by direct immersion into 4% paraformaldehyde (PFA) at 4° C. for 4 hours followed by dehydration and embedding in paraffin, and LCM was performed using the Veritas' Microdissection instrument Model 704 (ArcturusXT™, Molecular Devices, Applied Biosystems®, Life Technologies, Foster City, Calif., U.S.A.), as described in (Ernst E H et al., Dormancy and activation of human oocytes from primordial and primary follicles: Molecular clues to oocyte regulation. Hum Reprod. 2017 Aug. 1; 32(8):1684-1700). Isolates of oocytes, and oocytes with surrounding granulosa cells (follicles) from the primordial and primary stage, respectively (four main groups in total), from each of the three patients, a total number of 12 samples (Table 3), were isolated using LCM FIGS. 1A and B. RNA extraction, library preparation and sequencing, and bioinformatics were performed as previously described (Ernst E H et al., Dormancy and activation of human oocytes from primordial and primary follicles: Molecular clues to oocyte regulation. Hum Reprod. 2017 Aug. 1; 32(8):1684-1700)

TABLE 1
Number of patients and LCM-collected cells.

| | Number of oocytes | | Number of follicles | |
|---|---|---|---|---|
| | captured for each | | captured for each | |
| | follicle stage from | | stage from | |
| | the three patients | | the three patients | |
| Patient no. | Primordial (O) | Primary (O) | Primordial (F) | Primary (F) |
| 1 | 185 | 76 | 142 | 114 |
| 2 | 181 | 61 | 233 | 97 |
| 3 | 70 | 45 | 164 | 50 |
| Total | 436 | 182 | 539 | 261 |

(O) = oocytes,
(F) = follicles

### Animals

C57BL/6j×CBA F1 hybrid mice (Janvier Labs, France) were housed and bred in the animal facilities at Department of Biomedicine, Aarhus University. Animals were housed in a 12:12 h controlled light-dark environment and were provided with food and water ad libitum. 7-day-old female pups were used for the study.

Isolation of ovaries and organ culture Pups were sacrificed and ovaries were excised. Using a stereomicroscope MZ75 (Leica Microsystems, Germany), excess tissue was removed. During the dissection, ovaries were kept at 37° C. in culture medium: αMEM (Thermo Fisher) supplemented with 10% FBS (Thermo Fisher), 100 mIU/mL of FSH (Sigma-Aldrich), 100 IU/mL penicillin (Thermo Fisher), 100 μg/mL streptomycin (Thermo Fisher) and 1% Insulin-Transferrin-Selenium (Thermo Fisher). Isolated ovaries were transferred into well inserts (PET membrane ThinCert, 0.4 μm-pore size; Greiner bio-one) in 24-well plates (Tissue culture treated, cell culture plates; Costar). 200-300 μL of culture medium was added to the well below the insert and up to two ovaries were placed on the membrane of each insert. The culture medium is supplemented with different concentrations of compounds, listed in Table 2. Up to six wells in the 24-well plate were used for inserts. Sterile dH₂O was added to the rest of the wells to ensure humidity and reduce evaporation. The ovaries were cultured at 37° C., 5% CO₂ for four days. 150 μL of medium was replaced every other day with fresh culture medium.

TABLE 2

| | Target (protein) |
|---|---|
| | *¹Up-regulated |
| Compound | *²Down-regulated |
| Sotrastaurin | PRKCB |
| (antagonist) | protein kinase C beta |
| 3-(1H-indol-3-yl)-4-[2-(4-methylpiperazin-1- | |
| yl)quinazolin-4-yl]pyrrole-2,5-dione | |
| Ruboxistaurin | |
| (antagonist) | |
| 13-((dimethylamino)methyl)-10,11,14,15- | |
| tetrahydro-4,9:16,21-dimetheno-1H,13H- | |
| dibenzo(e,k)pyrrolo(3,4- | |
| h)(1,4,13)oxadiazacyclohexadecene-1,3(2H)- | |
| dione | |
| Trodusquemine | PTPN1*² |
| (antagonist) | protein tyrosine phosphatase, non- |
| [(3R,6R)-6- | receptor type 1 |
| [(3S,5R,7R,8R,9S,10S,13R,14S,17R)-3-[3-[4-(3 | |
| aminopropylamino)butylamino]propylamino | |
| ]-7-hydroxy-10,13-dimethyl- | |
| 2,3,4,5,6,7,8,9,11,12,14,15,16,17- | |
| tetradecahydro-1H | |
| cyclopenta[a]phenanthren-17-yl]-2- | |
| methylheptan-3-yl] hydrogen sulfate | |
| Claramine | |
| (3β,6β)-6-[[3-[[4-[(3- | |
| Aminopropyl)amino]butyl]amino]propyl]amino]- | |
| cholestan-3-ol trifluoroacetate salt | |
| Linagliptin | DPP4*¹ |
| (8-[(3R)-3-aminopiperidin-1-yl]-7-but-2-ynyl- | (Dipeptidyl Peptidase 4) |
| 3-methyl-1-[(4-methylquinazolin-2- | |
| yl)methyl]purine-2,6-dione) | |
| Vildagliptin | |
| ((2S)-1-[2-[(3-hydroxy-1- | |
| adamantyl)amino]acetyl]pyrrolidine-2- | |
| carbonitrile). | |
| Sitagliptin | |
| ((3R)-3-amino-1-[3-(trifluoromethyl)-6,8- | |
| dihydro-5H-[l,2,4]triazolo[4,3-a]pyrazin-7- | |
| yl]-4-(2,4,5-trifluorophenyl)butan-1-one). | |
| Talabostat | |
| ([(2R)-1-[(2S)-2-amino-3- | |
| methylbutanoyl]pyrrolidin-2-yl]boronic acid; | |
| methanesulfonic acid) | |
| Fasoracetam | GRM8*¹ |
| (agonist) | (glutamate metabotropic receptor |
| ((5R)-5-(piperidine-1-carbonyl)pyrrolidin-2- | 8) |
| one) | |
| NVP-LEQ-506 | SMO*¹ |
| (2-[5-[(2R)-4-(6-benzyl-4,5- | (smoothened, frizzled class |
| dimethylpyridazin-3-yl)-2-methylpiperazin-1- | receptor) |
| yl]pyrazin-2-yl]propan-2-ol) | |
| TAK-441 | |
| (6-ethyl-N-[1-(2-hydroxyacetyl)piperidin-4- | |
| yl]-1-methyl-4-oxo-5-phenacyl-3-(2,2,2- | |
| trifluoroethoxy)pyrrolo[3,2-c]pyridine-2- | |
| carboxamide) | |
| PF-04449913 | |
| 1-[(2R,4R)-2-(1H-benzimidazol-2-yl)-1- | |
| methylpiperidin-4-yl]-3-(4-cyanophenyl)urea | |
| Taladegib | |
| 4-fluoro-N-methyl-N-[1-[4-(2-methylpyrazol- | |
| 3-yl)phthalazin-1-yl]piperidin-4-yl]-2- | |
| (trifluoromethyl)benzamide | |
| Sonidegib | |
| (N-[6-[(2S,6R)-2,6-dimethylmorpholin-4- | |
| yl]pyridin-3-yl]-2-methyl-3-[4- | |
| (trifluoromethoxy)phenyl]benzamide) | |
| Vismodegib | |
| (antagonist) | |
| (2-chloro-N-(4-chloro-3-pyridin-2-ylphenyl)- | |
| 4-methylsulfonylbenzamide) | |
| Fluoxymesterone | PRLR*² |
| (antagonist) | Prolactin receptor |
| (8S,9R,10S,11S,13S,14S,17S)-9-fluoro-11,17- | |
| dihydroxy-10,13,17-trimethyl- | |
| 1,2,6,7,8,11,12,14,15,16- | |
| decahydrocyclopenta[a]phenanthren-3-one | |
| Pregnenolone | GABRE*² |
| (agonist) | gamma-aminobutyric acid type A |
| 1-[(3S,8S,9S,10R,13S,14S,17S)-3-hydroxy- | receptor epsilon subunit |
| 10,13-dimethyl- | |
| 2,3,4,7,8,9,11,12,14,15,16,17-dodecahydro- | |
| 1H-cyclopenta[a]phenanthren-17- | |
| yl]ethanone | |

### Histological Analysis and Follicle Counting

Ovaries kept in organ culture for four days were fixed for 24 hours in 4% paraformaldehyde solution at 4° C. After fixation, ovaries were dehydrated in ethanol series using 70%, 96% and 99.9% ethanol. Xylene was used as clearing agent before the ovaries were infiltrated in paraffin wax. 5 μm-sections of samples in paraffin were cut using a microtome (Cut 6062, SLEE medical, Germany). Paraffin sections were mounted on glass slides, paraffin was melted at 60° C., and the samples were stained with hematoxylin and eosin (using standard protocols).

The samples were deparaffinized by incubating in xylene for 2×15 min. and subsequently rehydrated in series of ethanol; 3×2 min. in 99.9% ethanol, 2 min. in 96% ethanol, and 2 min. in 70% ethanol. The samples were then rinsed in dH₂O, stained in hematoxylin for 40 sec., rinsed in dH₂O for 5 min., and stained in eosin for 46 sec. The samples were dehydrated in ethanol: 2×2 min. in 96% ethanol, 2×2 min in 99.9% ethanol, and finally cleared up in xylene for at least 30 min before mounting the samples using Eukitt mounting medium (Sigma-Aldrich) and cover glass.

The number of follicles at each developmental stage was counted using an inverted research microscope (DMI4000B, Leica Microsystems, Germany). The follicles of every 3^{rd} to 4^{th} section of each ovary were counted and the distribution of follicles in the different stages in percentage was determined. Every treatment was repeated on at least three biological repeats. Only follicles with a visible oocyte nucleus was counted. Follicles were classified as either primordial, primary and secondary. Briefly, primordial follicles consist of an oocyte encapsulated by flattened, squamous granulosa cells. Primary follicles are oocytes encapsulated by one layer of cuboidal granulosa cells, and secondary follicles consist of oocytes encapsulated by more than one layer of cuboidal granulosa cells.

### Statistical Analysis

The percentages of follicles in the different stages from each biological repeat were averaged. When comparing two groups, an unpaired t-test was performed. One-way ANOVA followed by determination of statistical significance using the Holm-Sidak method was performed when comparing more than two groups. Groups were considered significantly different if P≤0.05. Statistics were calculated with the help of GraphPad Prism (version 7.00 GraphPad Software, La Jolla, Calif., USA).

### Example 1: Identification of Candidates that are Involved in Follicle Development

### Data Preparation and Filtration of Granulosa Cell Transcriptome Data

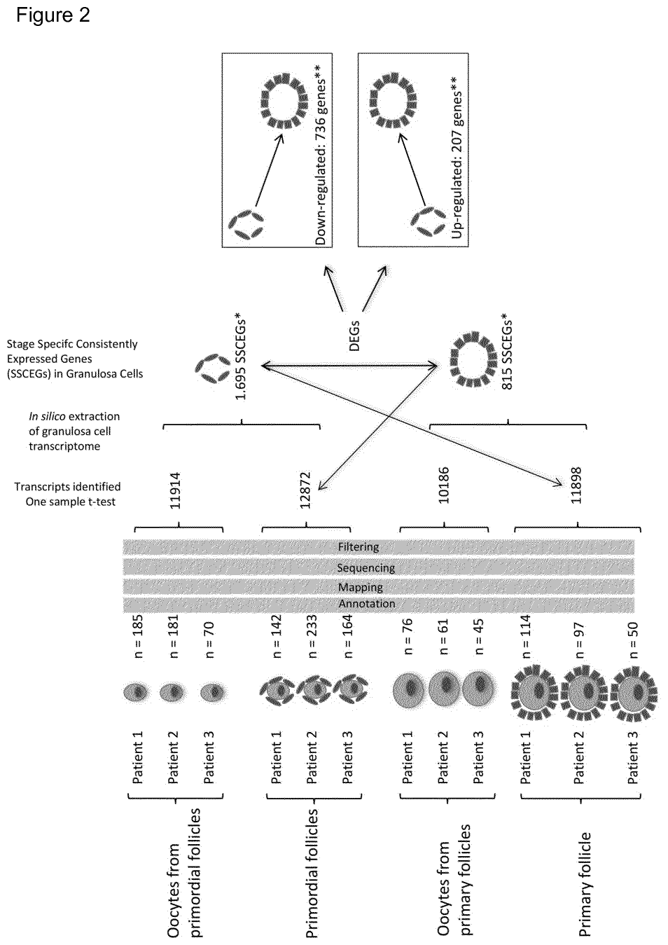

Pure isolated pools of oocytes from primordial and primary follicles, as well as whole follicles from the same stages were obtained using LCM (FIG. 1A) in tissue donated from three patients making up a total of 12 samples (three isolates of oocytes from primordial and primary follicles, respectively, and three isolates of primordial and primary follicles, respectively) (FIGS. 1B and 2) prior to library preparation and sequencing (FIG. 2). RNA-seq. yielded on average 35.3 million reads per sample (range: 31.8-39.6 million reads) and was mapped to the human genome (hg19) (average number of reads mapped: 31.7 million, range 29.4-34.0). Each gene was normalized and transformed to the binary logarithm value (log 2) prior to calculating fragments per kilo base of exon per million (FPKM) values (FIG. 1B).

Sorting and Enrichment Analysis of RNA Sequencing Data from Granulosa Cells from Primordial and Primary Follicles

Following RNA sequencing, mapping, and dataset filtration, the primordial oocyte transcriptome (11914 transcripts), primordial follicle transcriptome (12872 transcripts), primary oocyte transcriptome (10186 transcripts) and primary follicle transcriptome (11898 transcripts) (http://users-birc.au.dk/biopv/published_data/ernst_et_al_GC_2017/) were identified. In silico extraction of the granulosa cell transcriptome was performed (FIG. 2) and the Stage Specific Consistently Expressed Genes (SSCEGs) lists were defined for the granulosa cells from primordial and primary follicles (http://users-birc.au.dk/biopv/published_data/ernst_et_al_GC_2017/). The SSCEGs analysis of the granulosa cell transcriptome revealed 1695 transcripts (13.2%) (GC transcriptome contribution in primordial follicles) and 815 transcripts (6.8%) (GC transcriptome contribution in primary follicles) in primordial and primary follicles that fulfil these criteria (FIG. 2).

SSCEGs in granulosa cells from primordial and primary follicles were used to identify genes differentially expressed between the two cell populations. The DEGs analysis revealed 736 genes to be downregulated (highest expression in GCs from primordial follicles) and 207 genes to be upregulated (highest expression in GCs from primary follicles) in the granulosa cells during the primordial-to-primary follicle transition (FIG. 2). The heat map of DEGs FPKM values from the granulosa cells of primordial and primary follicles confirmed two different cell populations, and correlation between stage specific patient triplicates.

The specificity of the in silico extraction of the granulosa cell transcriptome from whole follicles was confirmed by the presence of a number of granulosa cell specific transcripts and absence of oocyte-specific transcripts in the SSCEG lists.

The continued Ingenuity® Pathway Analysis (IPA®)-generated enrichment analysis of the transcriptomes was performed on the following groups based on SSCEGs analysis: 1) SSCEGs in granulosa cells from primordial follicles (1.695 genes); 2) SSCEGs in granulosa cells from primary follicles (815 genes) (FIG. 2).

In the DEGs analysis, the following groups were analysed: (3) genes downregulated in granulosa cells from primordial to primary follicles (736 genes); and (4) genes upregulated in granulosa cells from primordial to primary follicles (207 genes) (FIG. 2).

Global Molecular Profiles in Granulosa Cells from Primordial Follicles

For granulosa cells from primordial follicles, a total of 1695 significant SSCEGs were subjected to IPA enrichment analysis, with the exception of 26 ENSEMBLE-IDs that remained un-mapped or filtered out during data uploading, leaving 1669 transcripts for further analysis.

Significantly enriched canonical pathways included ‘Autophagy’ (p=5.51E-04), which is involved in cellular degradation of redundant cytoplasmic molecules through actions of lysozymes. Other significantly enriched Canonical Pathways in granulosa cells of primordial follicles were ‘Androgen Signalling’ (p=3.97E-02) with 15 molecules assigned, among others the androgen receptor (AR), and the Canonical Pathways ‘Role of Oct4 in mammalian embryonic stem cell pluripotency’ (p=3.15E-02), ‘STAT3 pathway’ (p=1.75E-02) and ‘Corticotrophin Releasing Hormone Signalling’ (p=3.29E-02).

For detailed information on ‘Molecular and Cellular Functions’, ‘Biological Networks’ and ‘Canonical Pathways’, including which genes were assigned to each group in granulosa cells from primordial follicles SSCEGs.

Global Molecular Profiles in Granulosa Cells from Primary Follicles

For granulosa cell from primary follicles, a total of 815 significant SSCEGs (Resource Data 2) were subjected to IPA enrichment analysis, with the exception of 13 ENSEMBLE-IDs that remained un-mapped or filtered out during data uploading, leaving 802 for further analysis.

Granulosa cell genes downregulated in the primordial to primary transition 736 genes were significantly (paired t-test p<0.05 and/or >2-fold change) downregulated in granulosa cells from primary oocytes as compared to granulosa cells from primordial follicles (FIG. 2). 13 ENSEMBLE-IDs remained un-annotated or filtered out during uploading, thus leaving 723 for further analysis.

Granulosa cell genes upregulated in the primordial to primary transition Two hundred and seven genes were significantly (paired t-test p<0.05 and/or >2-fold change) upregulated in granulosa cells from primary oocytes as compared to granulosa cells from primordial follicles (FIG. 2). Seven ENSEMBLE-IDs remained un-annotated or filtered out during uploading, thus leaving 200 for further analysis.

Based on the transcriptome data the candidates mentioned in Table 3 were identified as candidates for use in regulating follicle development by adding a compound that changes the activity of the candidate and determining whether said compound was capable of regulating follicle maturation, in particular regulating the primordial to primary transitions of follicles.

TABLE 3

| | Primordial | Consistency | Primary | Consistency | | |
|---|---|---|---|---|---|---|
| | Granulosa | in primordial | granulosa | in primary | Paired t-test | |
| | cells, | granulosa | cells, | granulosa | across two | FOLD |
| | mean | cells FPKM | mean | cells FPKM | sets of | Change |
| Symbol | (FPKM) | value, t-test | (FPKM) | value, t-test | triplicates | UP |
| DPP4 | 0.895038265 | 0.422649731 | 4.330462254 | 0.007652781 | 0.022417116 | 4.838298457 |
| MAP1A | 0.113023906 | 0.422649731 | 3.965086657 | 0.198904214 | 0.204653733 | 35.08184076 |
| TLR3 | 0.586311652 | 0.422649731 | 3.774158439 | 0.183510216 | 0.196290496 | 6.437119964 |
| HDAC4 | 0.659224179 | 0.116619044 | 2.893986731 | 0.189383516 | 0.215360071 | 4.389988753 |
| POLE2 | 1.052051314 | 0.178636149 | 2.26430195 | 0.183842113 | 0.530528643 | 2.152273296 |
| GGCX | 0.254424469 | 0.278483529 | 2.105352985 | 0.186702407 | 0.183827206 | 8.274962663 |
| GRM8 | 0.938675923 | 0.353646083 | 1.957847156 | 0.155132427 | 0.438109973 | 2.0857541 |
| SMO | 0.617280498 | 0.183706582 | 1.929576642 | 0.189915216 | 0.211775674 | 3.125931646 |

| | Primordial | Consistency | Primary | Consistency | | |
|---|---|---|---|---|---|---|
| | Granulosa | in primordial | granulosa | in primary | Paired t-test | |
| | cells, | granulosa | cells, | granulosa | across two | FOLD |
| | mean | cells FPKM | mean | cells FPKM | sets of | Change |
| Symbol | (FPKM) | value, t-test | (FPKM) | value, t-test | triplicates | DOWN |
| JAK2 | 5.79159387 | 0.022491254 | 1.435800407 | 0.422649731 | 0.122912301 | 4.033704019 |
| COL12A1 | 4.836875848 | 0.005733454 | 1.716796746 | 0.408118497 | 0.137230377 | 2.817384096 |
| KCNB1 | 4.425819106 | 0.118215568 | 2.069541427 | 0.422649731 | 0.246011314 | 2.138550622 |
| PRLR | 4.190734628 | 0.078475807 | 1.337839787 | 0.185262895 | 0.141292175 | 3.132463745 |
| PRKCI | 4.139908578 | 0.027358974 | 1.999221112 | 0.410013617 | 0.225752511 | 2.070760734 |
| IL3RA | 3.971514007 | 0.001263048 | 1.33274011 | 0.265863062 | 0.11542358 | 2.979961341 |
| STS | 3.877953789 | 0.115808182 | 0.344697609 | 0.346419651 | 0.116431556 | 11.25030662 |
| CDK1 | 3.73201974 | 0.024477362 | 0.818236729 | 0.422649731 | 0.17084856 | 4.561051353 |
| NTRK2 | 3.692151106 | 0.026253405 | 1.254278686 | 0.422649731 | 0.094303565 | 2.943644939 |
| GANC | 3.608272812 | 0.060088885 | 0.414862739 | 0.422649731 | 0.060078107 | 8.697509981 |
| PSEN2 | 3.263341286 | 0.024146621 | 1.400426297 | 0.404532949 | 0.203847324 | 2.330248505 |
| PLCL1 | 2.980095604 | 0.065473835 | 0.927839068 | 0.214472796 | 0.224376281 | 3.211866914 |
| HDAC6 | 2.912450529 | 0.184471614 | 0.268294573 | 0.422649731 | 0.193157259 | 10.85542096 |
| FGFR2 | 2.873895326 | 0.138083172 | 0.106925131 | 0.422649731 | 0.132676779 | 26.87764137 |
| GABRE | 2.837021367 | 0.00215803 | 2.239821601 | 0.004355784 | 0.016471276 | 1.266628274 |
| PTPN1 | 2.835732044 | 0.040990075 | 0.951001956 | 0.422649731 | 0.150855344 | 2.981836183 |
| XDH | 2.709244397 | 0.132521709 | 0.694350157 | 0.422649731 | 0.280037462 | 3.901841698 |
| DCK | 2.46519817 | 0.088333685 | 1.005768937 | 0.422649731 | 0.123963903 | 2.45105817 |
| TSHR | 2.434836003 | 0.067802105 | 0.668443754 | 0.422649731 | 0.003986233 | 3.642544326 |
| LEPR | 2.406298361 | 0.18803688 | 0.388906925 | 0.422649731 | 0.245427129 | 6.187337394 |
| WEE1 | 2.267918596 | 0.187820419 | 0.721812363 | 0.422649731 | 0.305529008 | 3.141978042 |
| DBH | 2.174449998 | 0.035435187 | 0.815735018 | 0.391242864 | 0.360544067 | 2.665632773 |
| PRKCB | 2.157063354 | 0.112308895 | 1.238764037 | 0.235602011 | 0.00967475 | 1.741302854 |
| TUBD1 | 2.123487962 | 0.152299345 | 0.82762224 | 0.294464454 | 0.220413769 | 2.565769572 |
| PIM2 | 1.885118933 | 0.087718834 | 0.916657147 | 0.422649731 | 0.099225098 | 2.056514738 |
| BMI1 | 1.87595146 | 0.176182314 | 0.500214903 | 0.289993451 | 0.353032941 | 3.750291025 |
| XK | 1.68875363 | 0.184241651 | 0.617934348 | 0.422649731 | 0.313592619 | 2.732901377 |
| IMPDH1 | 1.630599782 | 0.187788769 | 0.546644414 | 0.422649731 | 0.318242858 | 2.982925905 |
| TUBA3C/TUBA3D | 1.624347348 | 0.18399001 | 0 | 0 | Significant | ∞ |
| ALOX12 | 1.58508984 | 0.183541266 | 0 | 0 | Significant | ∞ |
| CD274 | 1.537733901 | 0.193731745 | 0.056428242 | 0.422649731 | 0.216867791 | 27.25113973 |
| AOC3 | 0.514314059 | 0.18707281 | 0 | 0 | Significant | ∞ |
| CASP9 | 0.357300941 | 0.191902227 | 0 | 0 | Significant | ∞ |
| SLC52A2 | 0.220100615 | 0.183801318 | 0 | 0 | Significant | ∞ |
| ITGB7 | 0.116337219 | 0.183834169 | 0 | 0 | Significant | ∞ |
| LIPF | 0.116337219 | 0.183834169 | 0 | 0 | Significant | ∞ |

### Example 2: Evaluation of Follicle Numbers after Treatment with Compounds

### PRKCB

Protein kinase C (PKC) is a family of serine- and threonine-specific protein kinases that can be activated by calcium and second messenger diacylglycerol. PKC family members phosphorylate a wide variety of protein targets and are known to be involved in diverse cellular signaling pathways.

PKCα, PKCδ and PKCε isoforms was noted differentially expressed in particular cellular components of pre-pubertal, pubertal and adult mouse ovarian follicles Therefore, PKC isoforms will be examined for their ability to regulate follicular growth and oocyte maturation. If the results are positive, inhibition of PRKCB is a good target to prevent activation of primordial follicles.

### PTPN1

PTPN1 (Protein Tyrosine Phosphatase, Non-Receptor Type 1) mediates dephosphorylation of EIF2AK3/PERK by inactivating the protein kinase activity of EIF2AK3/PERK. This may include JAK2 and TYK 2 kinases. Therefore, it can be argued that inhibiting PTPN1 would allow JAK2 to translocate to the nucleus and activate growth factors. Thus, if the results are positive, pharmacological inhibition of PTPN1 can induce activation of primordial follicles.

### DPP4

DPP4 (Dipeptidyl Peptidase 4) is identical to adenosine deaminase complexing protein-2, and to the T-cell activation antigen CD26. It is an intrinsic membrane glycoprotein and a serine exopeptidase that cleaves X-proline dipeptides from the N-terminus of polypeptides. Linagliptin, a Dpp4 inhibitor, alleviated the phosphorylated protein expression of IGF/Akt/mTOR signaling pathway.

Thus, if the results are positive, pharmacological inhibition of DPP4 in ovarian follicle cells can lead to less phosphorylation of AKT/TOR pathway, which reduces the activation of primordial follicles.

### GRM8

GRM8 (Glutamate Metabotropic Receptor 8) is a G-protein coupled receptor for glutamate. Ligand binding causes a conformation change that triggers signaling via guanine nucleotide-binding proteins (G proteins) and modulates the activity of down-stream effectors, such as adenylate cyclase. Signaling inhibits adenylate cyclase activity. If the results are positive, Pharmacological inhibition of GRM8 will prevent activation of primordial follicles as adenylate cyclase activity is required to activate this process.

### SMO

SMO (Smoothened, Frizzled Class Receptor) and the protein encoded by this gene is a G protein-coupled receptor that interacts with the patched protein, a receptor for hedgehog proteins.

It has been shown that Smo deletion resulted in proteasomal degradation of the tumor suppressor PTEN and activation of oncogenic protein kinase B (AKT) in fibroblasts. Thus, if the results are positive, pharmacological inhibition of SMO in ovarian follicle cells will lead to activation of the AKT/TOR pathway, which would enhance the activation of primordial follicles.

### PRLR

PRLR (Prolactin Receptor) encodes a receptor for the anterior pituitary hormone, prolactin, and belongs to the type I cytokine receptor family. PRL receptor-deficient (PRLR(−/−)) female mice are sterile, despite ovulating due to a failure of embryo implantation, as a consequence of decreased ovarian LH receptor expression and inadequate corpus luteum formation and progesterone production. It is expected that pharmacological inhibition of PRLR would reduce the activation of primordial follicles.

### GABRE

GABRE (Gamma-Aminobutyric Acid Type A Receptor Epsilon Subunit) encodes the gamma-aminobutyric acid (GABA) A receptor, which is a multisubunit chloride channel that mediates the fastest inhibitory synaptic transmission in the central nervous system. This gene encodes an epsilon subunit. It has been shown that TGF-β1 enhances the binding of p53 and p65 to the GABRE (miR-224 host gene) promoter and then p53/p65 attenuate TGF-β1-mediated miR-224 transcription. The effects of miR-224 on inhibiting Smad4 expression and subsequently promoting GC proliferation and E2 release, were thus attenuated by the decreased miR-224 transcription.

Thus, it is expected that a pharmacological inhibition of GABRE might promote activation of primordial follicles.

### Items

The following items define preferred embodiments of the present invention:

1. A compound for use in regulating follicle maturation, wherein said compound can regulate the activity of Dipeptidyl Peptidase 4.
2. The compound according to item 1, wherein said compound is an inhibitor of Dipeptidyl Peptidase 4.
3. The compound according to item 2, wherein said inhibitor inhibits follicle maturation.
4. The compound according to any of items 2 and 3, wherein said inhibitor is selected from the group consisting of Saxagliptin, Alogliptin, Sitagliptin, Vildagliptin, Linagliptin and Talabostat.
5. A compound for use in regulating follicle maturation, wherein said compound can regulate the activity of toll like receptor 3.
6. The compound according to item 5, wherein said compound is an inhibitor of toll like receptor 3.
7. The compound according to item 6, wherein said inhibitor inhibits follicle maturation.
8. The compound according to any of items 6 and 7, wherein said inhibitor is Rintatolimod.
9. A compound for use in regulating follicle maturation, wherein said compound can regulate the activity of gamma-glutamyl carboxylase.
10. The compound according to item 9, wherein said compound is an inhibitor of gamma-glutamyl carboxylase.
11. The compound according to item 10, wherein said inhibitor inhibits follicle maturation.
12. The compound according to any of items 10-11, wherein said inhibitor is selected from the group consisting of Anisindione and Menadione.
13. A compound for use in regulating follicle maturation, wherein said compound can regulate the activity of glutamate metabotropic receptor 8.
14. The compound according to item 13, wherein said compound is an activator of glutamate metabotropic receptor 8.
15. The compound according to item 14, wherein said activator stimulates follicle maturation.
16. The compound according to any of items 13-14, wherein said activator is Fasoracetam.
17. A compound for use in regulating follicle maturation, wherein said compound can regulate the activity of smoothened, frizzled class receptor (SMO).
18. The compound according to item 17, wherein said compound is an inhibitor of smoothened, frizzled class receptor.
19. The compound according to item 18, wherein said inhibitor inhibits follicle maturation.
20. The compound according to any of items 18-19, wherein said inhibitor is selected from the group consisting of NVP-LEQ-506, TAK-441, PF-04449913, Taladegib, Sonidegib, Saridegib and Vismodegib.
21. A compound for use in regulating follicle maturation, wherein said compound can regulate the activity of potassium voltage-gated channel subfamily B member 1 (KCNB1).
22. The compound according to item 21, wherein said compound is an inhibitor of KCNB1.
23. The compound according to item 22, wherein said inhibitor stimulates follicle maturation.
24. The compound according to any of items 22-23, wherein said inhibitor is

Dalfampridine.

25. A compound for use in regulating follicle maturation, wherein said compound can regulate the activity of Prolactin receptor.
26. The compound according to item 25, wherein said compound is an inhibitor of Prolactin receptor.
27. The compound according to item 26, wherein said inhibitor inhibits follicle maturation.
28. The compound according to any of items 18-19, wherein said inhibitor is Fluoxymesterone.
29. A compound for use in regulating follicle maturation, wherein said compound can regulate the activity of cyclin dependent kinase 1.
30. The compound according to item 29, wherein said compound is an inhibitor of cyclin dependent kinase 1.
31. The compound according to item 30, wherein said inhibitor stimulates follicle maturation.
32. The compound according to any of items 30-31 wherein said inhibitor is selected from the group consisting of Dinaciclib, Milciclib, Roniciclib and Alvocidib.
33. A compound for use in regulating follicle maturation, wherein said compound can regulate the activity of neurotrophic receptor tyrosine kinase 2.
34. The compound according to item 33, wherein said compound is an inhibitor of neurotrophic receptor tyrosine kinase 2.
35. The compound according to item 34, wherein said inhibitor inhibits follicle maturation.
36. The compound according to any of items 34-35, wherein said inhibitor is selected from the group consisting of Cabozantinib, Theophylline and Entrectinib.
37. A compound for use in regulating follicle maturation, wherein said compound can regulate the activity of phospholipase C like 1.
38. The compound according to item 37, wherein said compound is an inhibitor of phospholipase C like 1.
39. The compound according to item 38, wherein said inhibitor inhibits follicle maturation.
40. The compound according to any of items 38-39, wherein said inhibitor is Quinacrine.
41. A compound for use in regulating follicle maturation, wherein said compound can regulate the activity of gamma-aminobutyric acid type A receptor epsilon subunit.
42. The compound according to item 41, wherein said compound is an activator of gamma-aminobutyric acid type A receptor epsilon subunit.
43. The compound according to item 42, wherein said activator stimulates follicle maturation.
44. The compound according to any of items 42-43, wherein said activator is selected from the group consisting of Eszopiclone and Pregnenolone.
45. A compound for use in regulating follicle maturation, wherein said compound can regulate the activity of protein tyrosine phosphatase, non-receptor type 1.
46. The compound according to item 45, wherein said compound is an inhibitor of protein tyrosine phosphatase, non-receptor type 1.
47. The compound according to item 46, wherein said inhibitor inhibits follicle maturation.
48. The compound according to any of items 46-47, wherein said inhibitor is Trodusquemine.
49. A compound for use in regulating follicle maturation, wherein said compound can regulate the activity of xanthine dehydrogenase.
50. The compound according to item 49, wherein said compound is an inhibitor of xanthine dehydrogenase.
51. The compound according to item 50, wherein said inhibitor inhibits follicle maturation.
52. The compound according to any of items 50-51, wherein said inhibitor is selected from the group consisting of Allopurinol, Oxypurinol and Febuxostat.
53. A compound for use in regulating follicle maturation, wherein said compound can regulate the activity of WEE1 G2 checkpoint kinase.
54. The compound according to item 53, wherein said compound is an inhibitor of WEE1 G2 checkpoint kinase.
55. The compound according to item 54, wherein said inhibitor inhibits follicle maturation.
56. The compound according to any of items 54-55, wherein said inhibitor is MK 1775.
57. A compound for use in regulating follicle maturation, wherein said compound can regulate the activity of protein kinase C beta.
58. The compound according to item 57, wherein said compound is an inhibitor of protein kinase C beta.
59. The compound according to item 58, wherein said inhibitor inhibits follicle maturation.
60. The compound according to any of items 58-59, wherein said inhibitor is selected from the group consisting of Sotrastaurin, Enzastaurin and Ruboxistaurin.
61. A compound for use in regulating follicle maturation, wherein said compound can regulate the activity of arachidonate 12-lipoxygenase, 12S type.
62. The compound according to item 61, wherein said compound is an inhibitor of arachidonate 12-lipoxygenase, 12S type.
63. The compound according to item 62, wherein said inhibitor inhibits follicle maturation.
64. The compound according to any of items 62-63, wherein said inhibitor is selected from the group consisting of Sulfasalazine, Balsalazide, Mesalamine and Masoprocol.
65. A compound for use in regulating follicle maturation, wherein said compound can regulate the activity of amine oxidase copper containing 3.
66. The compound according to item 65, wherein said compound is an inhibitor of amine oxidase copper containing 3.
67. The compound according to item 66, wherein said inhibitor inhibits follicle maturation.
68. The compound according to any of items 62-63, wherein said inhibitor is Hydralazine.
69. A compound for use in regulating follicle maturation, wherein said compound can regulate the activity of solute carrier family 52 member 2.
70. The compound according to item 69, wherein said compound is an inhibitor of solute carrier family 52 member 2.
71. The compound according to item 70, wherein said inhibitor inhibits follicle maturation.
72. The compound according to any of items 70-71, wherein said inhibitor is 4-hydroxybutanoic acid.
73. The compound according to any of the preceding items, for use in regulating follicle maturation in vitro.
74. The compound according to any of items 1 to 72, for use in regulating follicle maturation in vivo.
75. The compound according to any of the preceding items, wherein said compounds are for use in regulating primordial to primary transition of follicles.
76. The compound according to any of the preceding items, wherein said follicles are mammalian follicles.
77. The compound according to item 76, wherein said mammalian is a human.
78. A compound as defined in any of the preceding items for use in treating, preventing or ameliorating an ovulation disorder.
79. The compound according to item 61, wherein said ovulation disorder is selected from the group consisting of Polycystic ovary syndrome (PCOS), Premature ovarian failure (P01), Hypothalamic dysfunction and Menopause.
80. The compound according to item 61, wherein said ovulation disorder is caused by hyperprolactinemia.
81. A compound as defined in any of items 1 to 77 for use in treating, preventing or ameliorating infertility or reduced fertility in a female individual.
82. A pharmaceutical composition comprising at least one compound according to any of items 1 to 77 for use in treating, preventing or ameliorating infertility or reduced fertility in a female individual.
83. The pharmaceutical composition according to item 82, further comprising at least one pharmaceutically acceptable carrier.
84. The pharmaceutical composition according to any of items 82 to 83, further comprising an additional active agent.
85. The pharmaceutical composition according to any of items 82 to 84, wherein said composition is administered in vitro to primordial follicles from said individual.
86. The pharmaceutical composition according to any of items 82 to 85, wherein said female individual is a female mammal.
87. The pharmaceutical composition according to any of item 86, wherein said female mammal is a female human.
88. A method for treating, preventing or ameliorating infertility of a female individual having an ovulation disorder, comprising administering to said individual a therapeutically effective amount of a compound as defined in any of items 1 to 77.
89. The method according to item 88, wherein said female individual is a female mammal, such as a female human.
90. A method for screening for a compound that regulates follicle maturation, wherein said method comprises
a. selecting a compound known to regulate the activity of at least one of the candidates identified in Table 3.2.
b. contacting primordial ovaries with said compound
determining whether said compound is capable of regulating follicle maturation by determining the amount of primordial follicles and/or primary follicles and compare it with a control.

1. A compound for use in regulating follicle maturation, wherein said compound can regulate the activity of protein kinase C beta.

2. The compound according to claim 1, wherein said compound is an inhibitor of protein kinase C beta.

3. The compound according to claim 2, wherein said inhibitor inhibits follicle maturation.

4. The compound according to any of claims 1-3, wherein said inhibitor is selected from the group consisting of Sotrastaurin and Ruboxistaurin.

5. A compound for use in regulating follicle maturation, wherein said compound can regulate the activity of protein tyrosine phosphatase, non-receptor type 1.

6. The compound according to claim 5, wherein said compound is an inhibitor of protein tyrosine phosphatase, non-receptor type 1.

7. The compound according to claim 6, wherein said inhibitor inhibits follicle maturation.

8. The compound according to any of claims 5-7, wherein said inhibitor is Trodusquemine and Claramine.

9. A compound for use in regulating follicle maturation, wherein said compound can regulate the activity of Dipeptidyl Peptidase 4.

10. The compound according to claim 9, wherein said compound is an inhibitor of Dipeptidyl Peptidase 4.

11. The compound according to claim 10, wherein said inhibitor inhibits follicle maturation.

12. The compound according to any of claims 9-11, wherein said inhibitor is selected from the group consisting of Linagliptin, Vildagliptin, Sitagliptin and Talabostat.

13. A compound for use in regulating follicle maturation, wherein said compound can regulate the activity of glutamate metabotropic receptor 8.

14. The compound according to claim 13, wherein said compound is an activator of glutamate metabotropic receptor 8.

15. The compound according to claim 14, wherein said activator stimulates follicle maturation.

16. The compound according to any of claims 13-15, wherein said activator is Fasoracetam.

17. A compound for use in regulating follicle maturation, wherein said compound can regulate the activity of smoothened, frizzled class receptor (SMO).

18. The compound according to claim 17, wherein said compound is an inhibitor of smoothened, frizzled class receptor.

19. The compound according to claim 18, wherein said inhibitor inhibits follicle maturation.

20. The compound according to any of claims 17-19, wherein said inhibitor is selected from the group consisting of NVP-LEQ-506, TAK-441, PF-04449913, Taladegib, Sonidegib and Vismodegib.

21. A compound for use in regulating follicle maturation, wherein said compound can regulate the activity of Prolactin receptor.

22. The compound according to claim 21, wherein said compound is an inhibitor of Prolactin receptor.

23. The compound according to claim 22, wherein said inhibitor inhibits follicle maturation.

24. The compound according to any of claims 21-23, wherein said inhibitor is Fluoxymesterone.

25. A compound for use in regulating follicle maturation, wherein said compound can regulate the activity of gamma-aminobutyric acid type A receptor epsilon subunit.

26. The compound according to claim 25, wherein said compound is an activator of gamma-aminobutyric acid type A receptor epsilon subunit.

27. The compound according to claim 26, wherein said activator stimulates follicle maturation.

28. The compound according to any of claims 25-27, wherein said activator is selected from the group consisting of Pregnenolone.

29. The compound according to any of the preceding claims for use in regulating follicle maturation in vitro.

30. The compound according to any of the preceding claims for use in regulating follicle maturation in vivo.

31. The compound according to any of the preceding claims 1-28, wherein said compounds are for use in regulating primordial to primary transition of follicles.

32. The compound according to any of the preceding claims 1-28, wherein said follicles are mammalian follicles.

33. The compound according to claim 32, wherein said mammalian is a human.

34. A compound as defined in any of the preceding claims 1-28 for use in treating, preventing or ameliorating an ovulation disorder.

35. The compound according to claim 34, wherein said ovulation disorder is selected from the group consisting of Polycystic ovary syndrome (PCOS), Premature ovarian failure (POI), Hypothalamic dysfunction and Menopause.

36. The compound according to claim 34, wherein said ovulation disorder is caused by hyperprolactinemia.

37. A compound as defined in any of the preceding claims 1-28 for use in treating, preventing or ameliorating infertility or reduced fertility in a female individual.

38. A pharmaceutical composition comprising at least one compound according to any of the preceding claims for use in treating, preventing or ameliorating infertility or reduced fertility in a female individual.

39. The pharmaceutical composition according to claim 38, further comprising at least one pharmaceutically acceptable carrier.

40. The pharmaceutical composition according to any of claims 38 to 39, further comprising an additional active agent.

41. The pharmaceutical composition according to any of claims 38 to 40, wherein said composition is administered in vitro to primordial follicles from said individual.

42. The pharmaceutical composition according to any of claims 38 to 41, wherein said female individual is a female mammal.

43. The pharmaceutical composition according to any of claim 42, wherein said female mammal is a female human.

44. A method for treating, preventing or ameliorating infertility of a female individual having an ovulation disorder, comprising administering to said individual a therapeutically effective amount of a compound as defined in any of claims 1-28.

45. The method according to claim 44, wherein said female individual is a female mammal, such as a female human.

46. A method for screening for a compound that regulates follicle maturation, wherein said method comprises
g. selecting a compound known to regulate the activity of at least one of the candidates identified in Table 3.
h. contacting primordial ovaries with said compound determining whether said compound is capable of regulating follicle maturation by determining the amount of primordial follicles and/or primary follicles and compare it with a control.
